(19) United States
(12) Patent Application Publication
(10) Pub. No.: US 2026/0232684 A1
(43) Pub. Date: Aug. 13, 2026

(54) FLT3 INHIBITORS AND METHODS OF USING THE SAME

(71) Applicants: BIOVENTURES, LLC, Little Rock, AR (US); THE REGENTS OF THE UNIVERSITY OF CALIFORNIA, Oakland, CA (US)

(72) Inventors: Hong-yu LI, San Antonio, TX (US); Neil SHAH, Oakland, CA (US); Nick MCCONNELL, Oakland, CA (US)

(21) Appl. No.: 19/470,879

(22) PCT Filed: Apr. 1, 2024
(86) PCT No.: PCT/US2024/022492

§ 371 Date: Sep. 30, 2025

Related U.S. Application Data
(60) Provisional application No. 63/455,607, filed on Mar. 30, 2023.

(51) Int. Cl. A61K 31/5377; A61P 35/02; C07B 59/00; C07D 413/14

(52) U.S. Cl. CPC A61K 31/5377; A61P 35/02; C07B 59/002; C07D 413/14

(57) ABSTRACT

Disclosed are FMS like tyrosine kinase 3 inhibitors and methods of using the same for the treatment of a subject for a cell proliferative disorder.

### CROSS-REFERENCE TO RELATED APPLICATIONS

This application claims priority to U.S. provisional patent application No. 63/455,607, filed Mar. 30, 2023, the entirety of which is incorporated herein by reference.

### STATEMENT REGARDING FEDERALLY SPONSORED RESEARCH

This invention was made with government support under grant nos. R01 CA249282, R01 CA194094, and K00 CA212480, as awarded by the National Institutes of Health. The government has certain rights in the invention.

### BACKGROUND OF THE INVENTION

Efforts to optimize FLT3 inhibitors for the treatment of AML are ongoing (6). Despite having almost no activity as monotherapy, midostaurin was approved for patients with activating FLT3 mutations in 2017 after showing a small (7 percent) but significant improvement in overall survival when combined with chemotherapy compared to chemotherapy alone (7). However, midostaurin was not optimized to be a FLT3 inhibitor (8,9), and its exposure diminishes over time due to a poor pharmacokinetic profile. Quizartinib, the first potent FLT3 inhibitor, achieves deep remission in a substantial proportion of patients, and has been approved in Japan (10). However, development of secondary resistance occurs rapidly, most commonly due to resistance-conferring kinase domain mutations. Quizartinib is a “type II” kinase inhibitor (i.e. binds to an inactive “DFG-out” enzymatic conformation) (11). Type II inhibitors are susceptible to activation loop mutations in other class III receptor tyrosine kinases (PDFRA/B, KIT) (12). Additionally, profound myelosuppression is observed with all active FLT3 TKIs investigated to date. With quizartinib, myelosuppression is believed to be due to simultaneous inhibition of FLT3 and the closely related kinase KIT (13-16). Gilteritinib is a “type I” inhibitor (i.e. binds the active “DFG-in” conformation of FLT3) (15,17), and retains activity against quizartinib-resistant D835 mutations, although it remains vulnerable to resistance-conferring mutations at F691 (18). Gilteritinib also suffers from a sub-optimal safety profile and displays myelosuppression, likely due to a general lack of selectivity. Pexidartinib, a relatively selective Class III TKI recently approved for advanced tenosynovial giant cell tumors, has demonstrated efficacy against FLT3-ITD and F691L mutations but remains susceptible to D835 mutations, and is also associated with myelosuppression (19-21).

It is anticipated that the cure rate of FLT3-mutant AML will be substantially improved by potent FLT3 TKI maintenance therapy over a prolonged period. Optimized FLT3 TKIs must: (i) harbor sufficient potency against FLT3 to achieve disease response, (ii) retain activity against common clinically relevant resistant isoforms, and (iii) exhibit a high degree of kinase selectivity to minimize toxicity toward normal hematopoiesis. There are currently no approved or investigational TKIs that fully satisfy these requirements.

### BRIEF SUMMARY OF THE INVENTION

Disclosed are FMS like tyrosine kinase 3 inhibitors and methods of using the same for the treatment of a subject. One aspect of the invention provides for a compound having a formula of

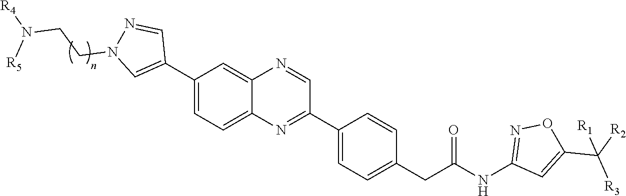

or a pharmaceutically acceptable salt thereof, wherein n is 1 or 2; R¹, R², and R³ are independently selected from methyl optionally substituted with a halo; and R⁴ and R⁵ together form a heterocyclic ring or C₁-C₃ alkyl. An exemplary compound is

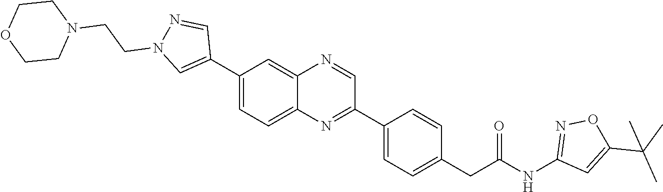 [SMILES: CC(C)(C)c1cc(NC(=O)Cc2ccc(-c3cnc4cc(-c5cnn(CCN6CCOCC6)c5)ccc4n3)cc2)no1]

Another aspect of the technology provides for a method of treatment. The method may comprise treating a subject for a cell proliferative disorder by administering a FMS like tyrosine kinase 3 inhibitor to the subject. The cell proliferative disorder may be a leukemia, such as acute myeloid leukemia (AML). AMLs that may be treated include those characterized by a FLT3 mutation. Exemplary FLT3 mutations include D835 and F691 mutations.

The FMS like tyrosine kinase 3 inhibitor used in the methods disclosed herein may comprise a compound having a formula of

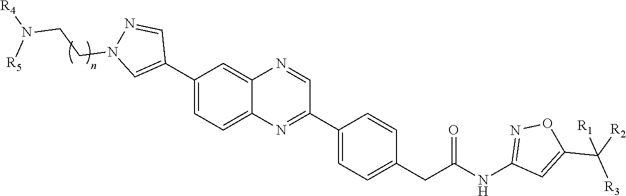

or a pharmaceutically acceptable salt thereof, wherein n is 1 or 2; R¹, R², and R³ are independently selected from methyl optionally substituted with a halo; and R⁴ and R⁵ together form a heterocyclic ring or C₁-C₃ alkyl. An exemplary compound for use in the method is

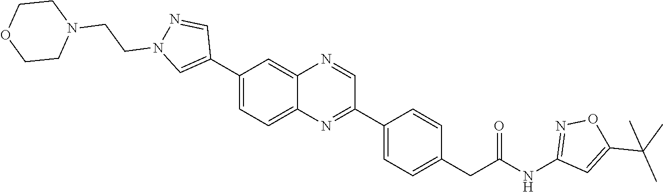 [SMILES: CC(C)(C)c1cc(NC(=O)Cc2ccc(-c3cnc4cc(-c5cnn(CCN6CCOCC6)c5)ccc4n3)cc2)no1]

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Activity of compounds 1 and 2 against parental Molm14 cells harboring secondary kinase domain mutations associated with clinical resistance to active FLT3 TKIs. Panel (A) shows calculated IC₅₀ values for gilteritinib, quizartinib, 1 and 2 in mutant FLT3 MV-4-11 and Molm14 cell lines and non-mutant FLT3 HL60 and K562 cell lines. Each point represents the calculated IC₅₀ from an independent experiment completed in triplicate. Cells were treated with inhibitor at increasing log doses (0.1 nM-10 μM) for 48 hours. Cell viability was analyzed through CellTiter GLO. (B-C) Western blot analysis using anti-phospho-tyrosine (top panel) and anti-FLT3 antibodies (second panel) on FLT3 immunoprecipitates and phospho-ERK, ERK, phospho-STAT5, STAT5, phospho-S6, S6 and GAPDH antibodies performed on whole cell lysates of Molm14, Molm14 D835Y, and Molm14 F691L cells treated with Gilteritinib, Quizartinib, Panel (B) shows compound 1 and Panel (C) shows compound 2. Cells were exposed to increasing amounts of each drug for 2 hours. GAPDH1 represents loading control for phospho-ERK and ERK blots. GAPDH2 represents loading control for phosphor-STAT5, STAT5, phospho-S6 and S6. Each experiment was completed in triplicate. Representative images are shown.

FIG. 2. KINOMEscan analysis and CFU assays of compounds 1, 2 and gilteritinib. Panel (A) shows KinomeScan results for gilteritinib, 1 and 2. Each drug was screened against a panel of 468 kinases and kinase mutants at two dosage points. Panel (B) shows calculated percentage of colonies compared to control for gilteritinib 1 and 2. Normal bone marrow mononuclear cells (BM-MNC) were purchased from StemCell Technologies. 30,000 cells were plated in methylcellulose with increasing concentrations of either gilteritinib, 1, or 2. Blast forming unit-erythroid (BFU-E) and colony forming unit-granulocyte (CFU-G) were counted after 14 days incubation. Assay was completed in triplicate with three independent blood donors. Data shown is cumulated from all independent experiments. Error bars show one standard deviation and p values compared to control were calculated using a Student's t-test. NS indicates an insignificant p value, * indicates p value≤0.05, ** indicates p value≤0.01, *** indicates p value≤0.001, **** indicates p value≤0.0001

FIG. 3. Molecular docking study of compound 1 and impact of the most common gatekeeper variants in the human kinome on activity toward FLKT. Panel (A) shows the structure of compound 1. Panel (B) shows compound 1 bound to FLT3 active model showing the dimethylamine group in close proximity to E661, F691 and F830. The dim FLT3 active model was built using 1PKG (KIT) as a reference using Prime (Schrodinger®). The complex of active FLT3 bound to 1 was minimized by Embrace (Schrodinger®). Panel (C) shows representative IC₅₀ results comparing the activity of 1 in BaF3 FLT3-ITD and BaF3 cell lines harboring FLT3-ITD with gatekeeper mutations representing other naturally occurring residues across the kinome. Cells were treated with inhibitor at increasing log doses (0.1 nM-10 μM) for 48 hours. Cell viability was analyzed through CellTiter GLO.

FIG. 4. Computational modeling of compound 2 and quizartinib. (A) Chemical structures of 2 and quizartinib highlighting the key carbon/nitrogen substitution. (B) Superimposition between models of FLT3 bound to 2 (beige), quizartinib (pink) and an active FLT3 model (yellow). FLT3 active model was built using 1PKG (KIT) as a reference using Prime (Schrodinger®). (C) Difference in MM/GBSA energies of quizartinib and 2 complexed with FLT3 (pink and beige) and FLT3 F691L (green and blue). Complexes were minimized by Embrace (Schrodinger®) followed by MM/GBSA calculations Prime (Schrodinger®) allowing flexibility of residues 5 Å from the ligands.

FIG. 5. Impact of linker swapping on activity of compound 2 and quizartinib. (A) Chemical structures of 2, quizartinib, and their respective analogs with the linker substitution highlighted. (B) Calculated IC₅₀ values for 2, 2 C—N, quizartinib, and quizartinib N—C in mutant FLT3-driven Molm14 cell lines. The values above the Molm14 D835Y and Molm14 F691L IC₅₀ values represent the fold increase in IC₅₀ compared to the parental Molm14 IC50. Each point represents the calculated ICso from an independent experiment completed in triplicate. Cells were dosed with inhibitor at increasing log doses (0.1 nM-10 μM) for 48 hours. Cell viability was analyzed through CellTiter GLO.

FIG. 6. Assessment of in vivo target inhibition by compounds 1 and 2 in Molm14 subcutaneous xenografts. Western blot analysis of 1 and 2 in a Molm14 xenograft model. Mice were dosed at one of three concentrations of either drug and tumors were excised at either 4 hours, 8 hours, or 24 hours, flash frozen and probed for inhibition of FLT3 signaling.

FIG. 7. In vivo efficacy of compound 2 and quizartinib against Molm14 F691L in vivo. (A) 14 day tumor growth based on bioluminescence measurement of mice dosed daily by oral gavage with either vehicle control, 5 mg/kg or 10 mg/kg of 2 or 33 mg/kg of gilteritinib, starting 14 days after tail vein injection. Molm14 F691L stably expressing FLuc9 were engrafted in mice through tail vein injection. Mice were imaged once every three days. Error bars show one standard deviation and p values compared to control were calculated using a Student's t-test. * indicates p value≤0.05, ** indicates p value≤0.005. (B) Bioluminescence imaging of tumor burden in each mouse dosed with either vehicle control, 5 mg/kg or 10 mg/kg of compound 2 or 33 mg/kg of gilteritinib at day 3 of dosing and day 14 of dosing.

FIG. 8. Activity of compounds against mutant FLT3 transduced cell lines. Calculated IC₅₀ values for quizartinib, gilteritinib 1 and 2 in Ba/F3 transduced cell lines. Each point represents the calculated IC₅₀ from an independent experiment completed in triplicate. Cells were treated with inhibitor at increasing log doses (0.1 nM-10 μM) for 48 hours. Cell viability was analyzed through CellTiter GLO.

FIG. 9. Impact of compounds on target engagement and downstream signaling in Ba/F3 cells expressing secondary mutations in FLT3-ITD. (A-D) Western blot analysis of phospho-FLT3 and FLT3 as well as downstream signaling proteins of phospho-ERK, ERK, phospho-STAT5, STAT5, phospho-S6, S6 and GAPDH performed on whole cell lysates of BaF3 cells transduced with (A) FLT3 ITD, (B) FLT3 D835V, (C) FLT3 ITD+D835V, (D) and FLT3 ITD+F691L comparing 1, 2, gilteritinib, and quizartinib. Cells were exposed to increasing amounts of each drug for 2 hours. GAPDH1 represents loading control for phospho-ERK and ERK blots. GAPDH2 represents loading control for phosphor-STAT5, STAT5, phospho-S6 and S6. Each experiment was completed in triplicate. Representative images are shown.

FIG. 10. (A) Schematic depicting the saturation mutagenesis experimental design and the range of concentrations for compound 1 and compound 2 to select for resistant cells; (B) Population doublings of XL1-Red-mutagenized FLT3-ITD plasmid-transduced Ba/F3 cells in escalating concentrations of compound 1 and compound 2. Arrows indicate the timepoint of passage when a concentration of 0.1 million cells/mL was achieved; (C) Western blot analysis of lysates collected from transduced Ba/F3 cells cultured in (lane 1) vehicle; (lane 2) 120 nM of compound 1; (lane 3) 120n M of compound 2 (lane 4) Ba/F3 cells transduced with MSCVpuro; (lane M) size marker. Western blot quantification indicates that FLT3-ITD is 28.89× and 17.11× overexpressed in 120 nM drug concentrations for compounds 1 and 2, respectively, compared with cells propagated in vehicle when normalized to GAPDH. Left table: identity of mutations detected at escalating concentrations of compounds 1 and 2. Right table: IC50 values of compounds 1 and 2 in Ba/F3 cells transformed to growth factor independence with FLT3-ITD and various mutant isoforms.

FIG. 11. Activity of compounds against primary FLT3-ITD AML samples in short term liquid culture assays. Cell viability measurements of five FLT3 mutant patient samples dosed with either vehicle control or 100 nM of quizartinib, gilteritinib, 1, or 2. Plates were incubated with drug for 0, 1, 2, 3, 5 or 7 days. FLT3 mutation is noted for each patient in parentheses. Cell viability was analyzed through CellTiter GLO.

FIG. 12. Activity of compound 1 against the NCI-60 cell line panel. Percentage growth of 1 in the 30 μM single dose experiment within the NCI-60 cell line panel. No cell line within the panel harbors a FLT3 mutation.

FIG. 13. Activity of compound 2 against the NCI-60 cell line panel. Growth inhibition curves of 2 in the 5-dose GIso experiment within the NCI-60 cell line panel. No cell line within the panel harbors a FLT3 mutation.

FIG. 14. Enzyme kinetic analysis of compound 1 and 2 against FLT3 and FLT3 D835Y. The 5-FAM labeled microfluidic assay was utilized and all the activities were measured using Caliper EZ Reader II with a 12-sipper chip. In the presence of variable concentrations of compound 1 or 2, phosphorylation activities of FLT3 and FLT3 D835Y were measured at different level of ATP within certain time durations. (A) The inhibition kinetic of compound 1 on FLT3, which indicated that compound 1 was a type 1 inhibitor of FLT3. (B) The inhibition kinetic of compound 2 on FLT3, which revealed that compound 2 was a type 2 inhibitor of FLT3. (C) The inhibition kinetic of compound 2 on FLT3 D835Y, which showed that the inhibition model was between type 1 and 2.

### DETAILED DESCRIPTION OF THE INVENTION

Disclosed herein are FMS like tyrosine kinase 3 (FLT3) tyrosine kinase inhibitors (TKIs). Cellular studies demonstrate that the disclosed inhibitors are potently and selectively active against FLT3-ITD.

Acute myeloid leukemia (AML) causes death within two years following diagnosis in the majority of cases. Approximately 10,000 people in the USA die annually of AML (1). Notably, the number of coding mutations per AML genome is low compared to other cancers (2). Optimized targeted therapy against critical targets therefore has considerable promise in AML. FLT3 receptor is the most commonly mutated gene in AML (~30% of cases). Most mutations occur in the juxtamembrane domain as internal tandem duplication (ITD) mutations, which confer a poor prognosis (3). FLT3-ITD results in ligand-independent kinase activation and downstream signaling through the RAS/RAF/MEK/ERK, JAK/STAT, and PI3K/AKT pathways, which ultimately promotes uncontrolled growth and survival. Less commonly, activating mutations occur within the activation loop of the kinase, typically at D835 (4).

The disclosed compounds retain activity against both secondary D835 and F691 mutant isoforms while nearly completely avoiding suppression of normal hematopoiesis in vitro. Broader kinome studies illustrate their improved selectivity toward FLT3. The compounds achieve prolonged target inhibition in a murine subcutaneous xenograft model of FLT3-ITD+ AML following a single dose. As demonstrated in the Examples, Compound 2 is more active than gilteritinib in a circulating cell line-derived xenograft model of FLT3-ITD+ AML in mice. Molecular docking, and dynamics and enzyme kinetic studies provide structural insights into the potency and selectivity of these compounds, in addition to their ability to retain activity against problematic TKI-resistant mutants.

FLT3 is the most commonly mutated gene in acute myeloid leukemia (AML). High rates of primary and secondary resistance, as well as poor therapeutic windows resulting in dose-limiting myelosuppression have minimized their impact on clinical outcomes. The selective FLT3 TKIs disclosed herein are characterized with distinct binding modes that are capable of maintaining efficacy across problematic mutant isoforms and sparing suppression of normal hematopoiesis are required to achieve a more substantial clinical impact. Moreover, they appear to be invulnerable to resistance-conferring mutations in FLT3. Mechanistic studies based upon computational modeling and enzyme kinetic studies provide insights into how these TKIs overcome two common and critical TKI challenges: increasing selectivity through mimicking biological interactions and improving activity against on-target resistance mutations through novel inhibitor structural modifications.

In some embodiments, the TKI described herein comprise a compound of formula

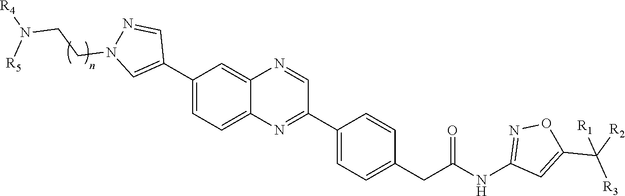

or a pharmaceutically acceptable salt thereof, wherein n is 1 or 2; R¹, R², and R³ are independently selected from methyl optionally substituted with a halo; and R⁴ and R⁵ together form a heterocyclic ring or C₁-C₃ alkyl.

The terms “heterocyclyl” and “heterocyclic ring” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. Unless specified otherwise, the heterocyclic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido or carboxyamido, carboxylic acid, —C(O) alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. Examples of heterocyclic rings include those having nitrogen and oxygen, such as a morpholine ring

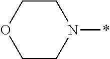 [SMILES: *N1CCOCC1]

The term “alkyl” as contemplated herein includes a straight-chain or branched alkyl radical in all of its isomeric forms, such as a straight or branched group of 1-12, 1-10, or 1-6 carbon atoms, referred to herein as C₁-C₁₂ alkyl, C₁-C₁₀-alkyl, and C₁-C₆-alkyl, respectively. Unless specified otherwise, the alkyl may be substituted at one or more positions with, for example, halogen, azide, alkyl, aralkyl, alkenyl, alkynyl, cycloalkyl, hydroxyl, alkoxyl, amino, nitro, sulfhydryl, imino, amido or carboxyamido, carboxylic acid, —C(O) alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. An example is a methyl optionally substituted with one or more halo, such as CH₃, CH₂F, CHF2, or CF₃.

Exemplary TKI include, without limitation,

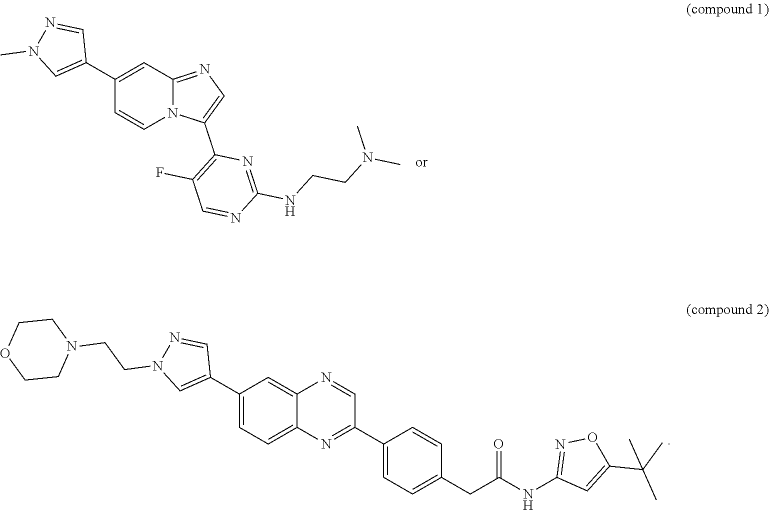

In some embodiments, the compounds of the disclosure may contain one or more chiral centers and, therefore, exist as stereoisomers, such as enantiomers or diastereomers. As used herein, the term “stereoisomers” refers to the enantiomers or diastereomers of a compound. These compounds may be designated by the symbol “R” or “S,” depending on the configuration of substituents around the stereogenic carbon atom. Mixtures of enantiomers or diastereomers may be designated “(±)” in nomenclature, but the skilled artisan will recognize that a structure may denote a chiral center implicitly. It is understood that the generic chemical structures (e.g. Formula I) encompass all stereoisomeric forms of the specified compounds, unless indicated otherwise.

In some embodiments, the compounds as described herein can be provided as pharmaceutically acceptable salts. As used herein, the term “pharmaceutically acceptable salt” refers to salts prepared by reaction of the compounds as disclosed herein with a pharmaceutically acceptable mineral or organic acid or an organic or inorganic base. Such salts are known as acid addition and base addition salts. The particular counterion forming a part of any salt of a compound disclosed herein may not be critical to the activity of the compound, so long as the salt as a whole is pharmacologically acceptable and as long as the counterion does not contribute undesired qualities to the salt as a whole. Undesired qualities may include undesirably solubility or toxicity.

The present disclosure includes all pharmaceutically acceptable isotopologues or isotopically-labelled compounds, wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of may generally be prepared by conventional techniques known to those skilled in the art or by processes analogous to those described in the accompanying Examples using an appropriate isotopically-labeled reagents in place of the non-labeled reagent previously employed.

Isotopologues with a designated isotope at a specific position also contain the isotope that predominates in nature at that position; the exact amount depends on the isotopic enrichment factor. It should also be understood that, unless otherwise stated, compounds with designated isotopes in a specific position contain the isotope at an abundance of at least 50% of its natural isotopic composition. The term “isotopic enrichment factor” refers to the ratio of the amount of a particular isotope in an enriched compound to the known natural amount of the isotope. The isotopic enrichment factor can be described as, for example, % deuterium/.0156 (natural abundance). A compound with 50% deuterium at a specific position would have an isotopic enrichment factor of 50/.0156=3205. Unless otherwise stated, compounds with designated deuterium in a specific position contain deuterium at an abundance of at least 3205 (50%) of its natural isotopic composition. Suitably the isotope enrichment factor may be at least 60%, 70%, 80%, 90%, or 95% of the isotopes natural isotopic composition.

The application also provides a pharmaceutical composition. The pharmaceutical composition comprises a compound as described herein, or a pharmaceutically acceptable salt thereof, and further comprises a pharmaceutically acceptable excipient, diluent, or carrier.

The pharmaceutical composition may comprise an effective amount of a compound as described herein. As used herein, the term “effective amount” refers to the amount or dose of the compound that provides the desired effect, upon single or multiple dose administration to the subject. A skilled artisan would understand that an effective amount can be readily determined by the attending diagnostician by the use of known techniques and by observing results obtained under analogous circumstances.

As used herein, the term “pharmaceutically acceptable excipient, diluent, or carrier” refers to a material that can be used as a vehicle for administering a therapeutic or prophylactic agent, (e.g., the compounds as described herein), because the material is inert or otherwise medically acceptable, as well as compatible with the agent. Such pharmaceutical compositions may be prepared by any method known in the art of pharmacy, for example by bringing into association the active ingredient with the carriers or excipients.

In some embodiments, the compound or the pharmaceutical composition as described herein is administered intraperitoneally, topically, and/or orally. Examples of pharmaceutical compositions containing the compounds for oral administration include capsules, syrups, concentrates, powders and granules. Examples of pharmaceutical compositions containing the compounds for intraperitoneal administration include suspensions, concentrates, or solutions. A suitable solvent system for preparing suspensions, concentrates, or solutions of the compounds includes the combination of DMSO, PEG300, Tween80, and saline or water in any equivalents and sequences.

In some embodiments, the compounds disclosed herein may be formulated as pharmaceutical compositions that include an effective amount of one or more compounds as disclosed herein and one or more pharmaceutically acceptable carriers, excipients, or diluents. In some embodiments, pharmaceutical compositions as described herein comprise substantially purified diastereomers or enantiomers of the compounds as disclosed herein (e.g., a composition comprising at least about 90%, 95%, or 99% pure diastereomer or enantiomer).

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder.

As used herein, a “subject” may be interchangeable with “patient” or “individual,” which may be a human or a non-human animal in need of treatment. In some embodiments, the subject in need of treatment may include a subject having a cell proliferative a cell proliferative disorder, such as a cancers. The method comprises administering an effective amount of the compounds as described herein, or a pharmaceutically acceptable salt thereof, or the pharmaceutical compositions as described herein, to a subject having the cell proliferative disorder. The subject may suffer from a leukemia, such as acute myeloid leukemia. The cell proliferative disorder may be characterized by a FLT3 mutation, such as a D835 or F691 mutation. Examples include D835Y and F691L mutations.

Another aspect of the invention provides for a method for reducing or inhibiting cancer cell growth in a subject having cancer. The method comprises administering an effective amount of the compounds as described herein, or a pharmaceutically acceptable salt thereof, or the pharmaceutical compositions as described herein, to the subject having cancer.

The compound may be administered alone or in combination with another therapy. Examples include a cell proliferative disorder chemotherapy, such as a chemotherapy for AML. A co-administered therapy may be provided before, concurrently with, or after administration of a compound described herein. In some embodiments, the TKI may be co-administered with another therapy for a period of time and the secondary therapy discontinued while the TKI is administered as a maintenance therapy for months or years longer. The TKIs described herein advantageously limit myelosuppression thereby allowing for longer maintenance therapy without the deleterious side-affects often associated with TKIs. Exemplary AML chemotherapies that may be administered with the compounds described herein include, without limitation, nucleoside analogs, anthracyclines, hypomethylating agents, BCL2 antagonists, antracenediones, RAS inhibitors, MEK inhibitors, IDH1/2 inhibitors, MDM2 inhibitors, menin inhibitors, antibody-based therapies and immunotherapies, and chimeric antigen receptor modified cellular therapy.

The application also provides a method of making the FLT3 inhibitors disclosed herein, including a compound corresponding to the structure:

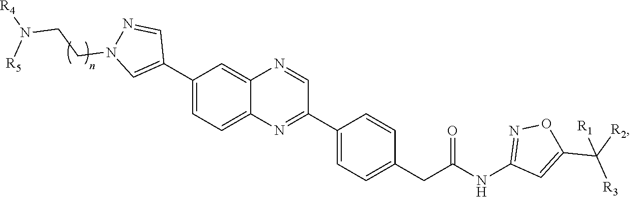

or a pharmaceutically acceptable salt thereof, wherein n is 1 or 2; R¹, R², and R³ are independently selected from methyl optionally substituted with a halo; and R⁴ and R⁵ together form a heterocyclic ring or C₁-C₃ alkyl. The compound may be prepared by converting a compound of Formula III

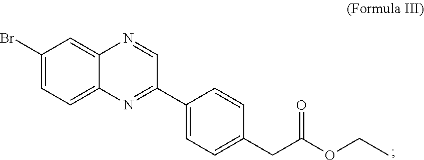 [SMILES: CCOC(=O)Cc1ccc(-c2cnc3cc(Br)ccc3n2)cc1]

to a compound of Formula II

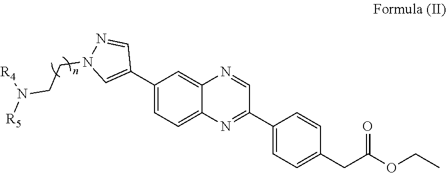

and further to a compound of Formula I

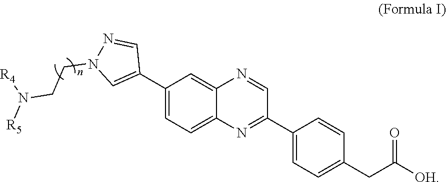

Formula I can be converted to the FLT3 inhibitor. Exemplary methods and conditions for converting the compounds are disclosed in the Synthesis of Compound 2 of the Examples below.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Compound 1 and Compound 2 Display Improved Comprehensive Activity Against FLT3-ITD and Secondary Mutations

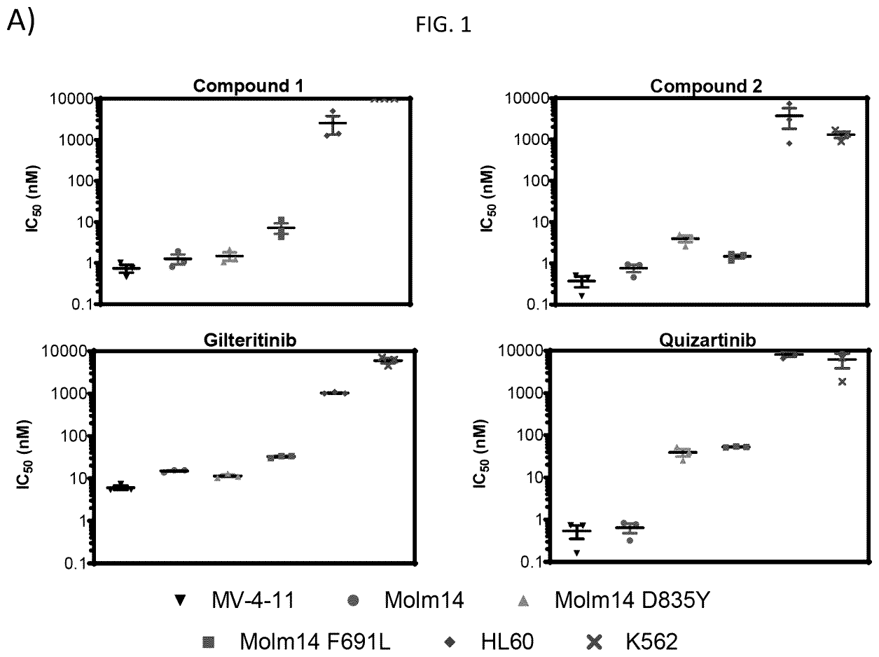

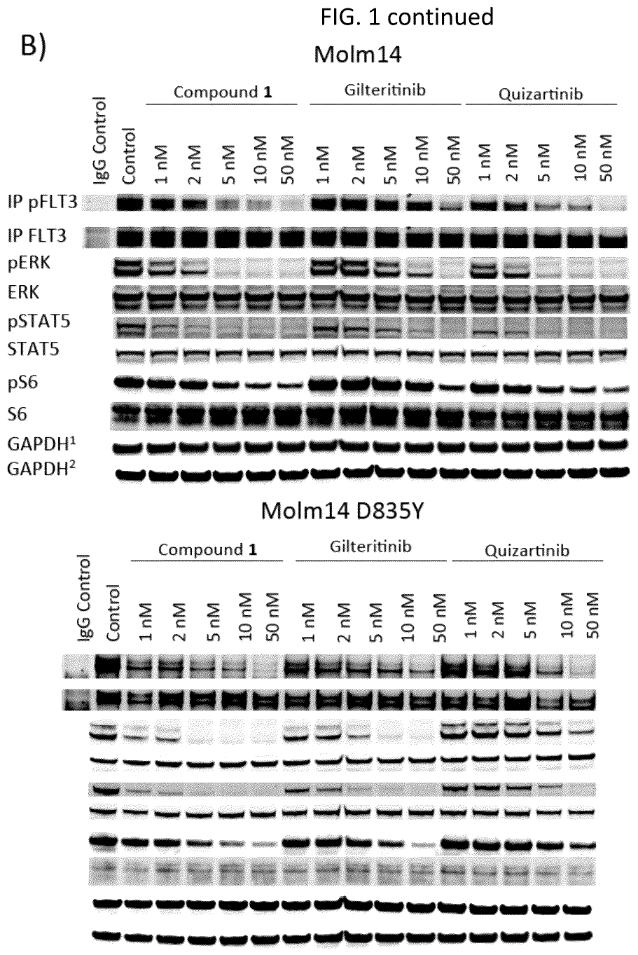

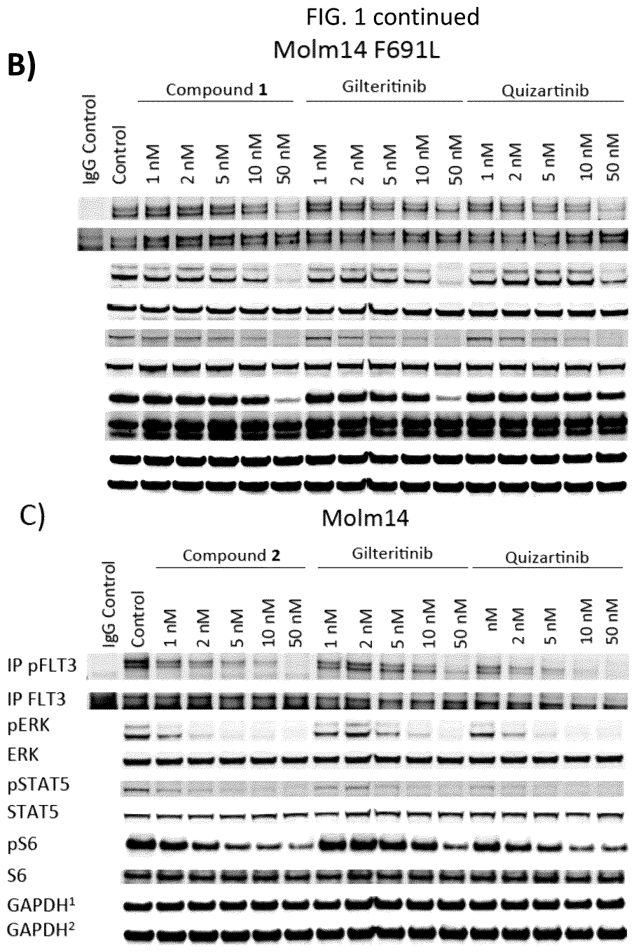

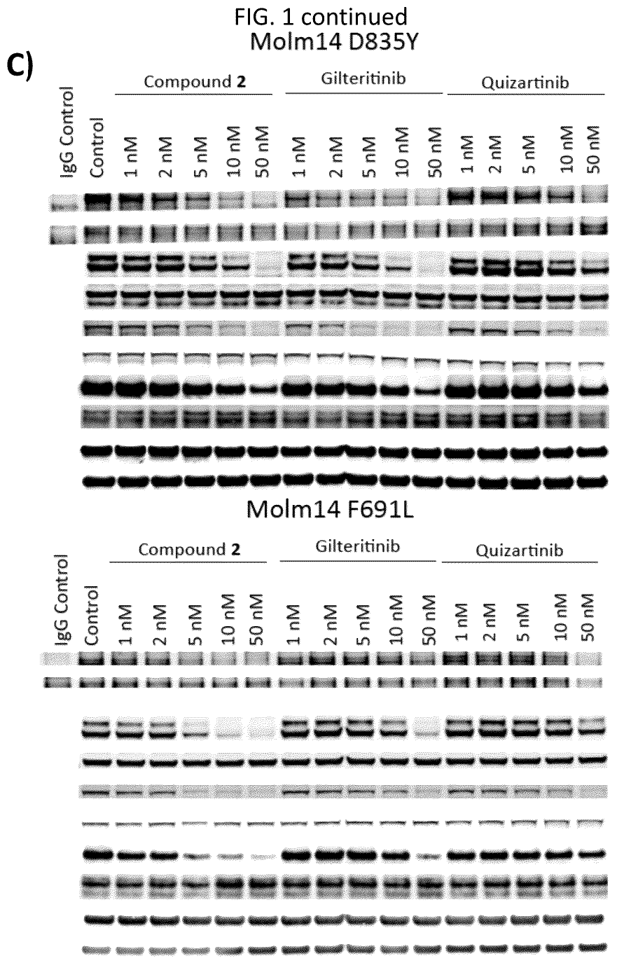

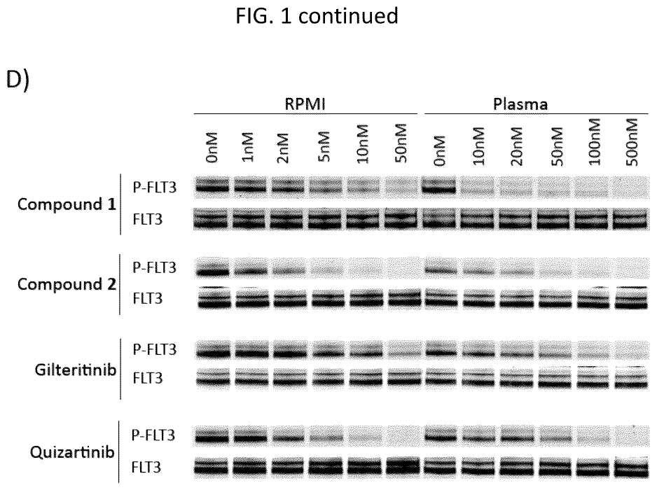

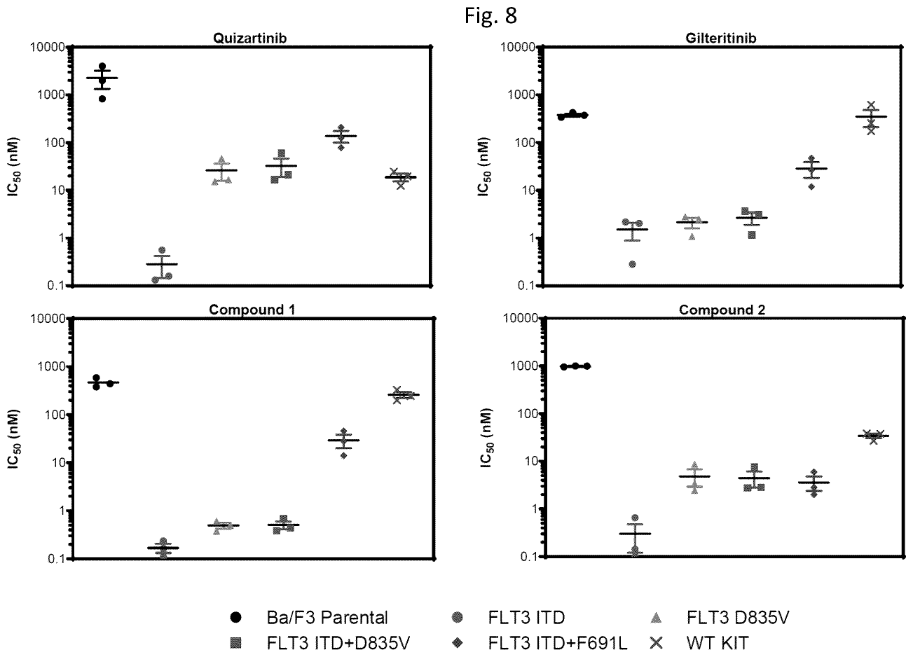

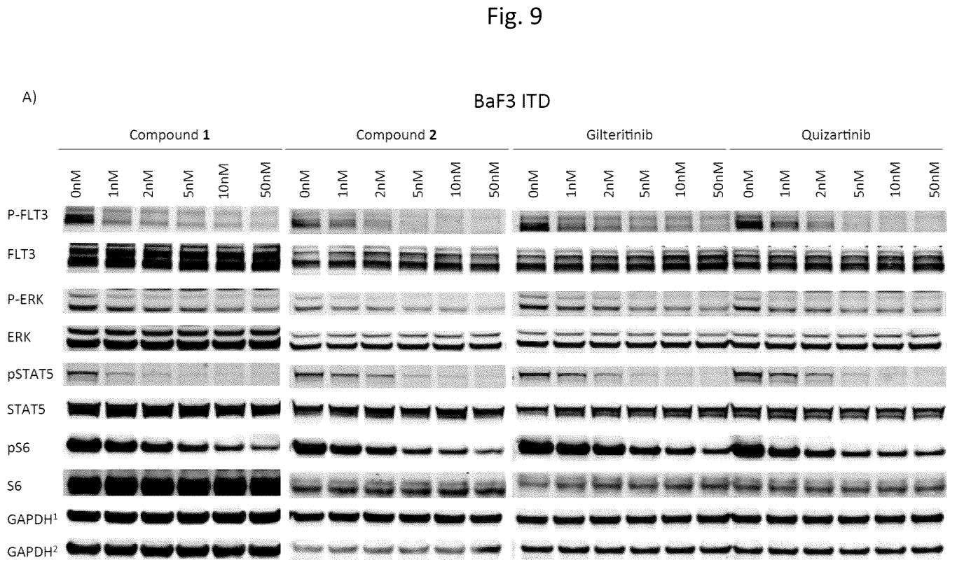

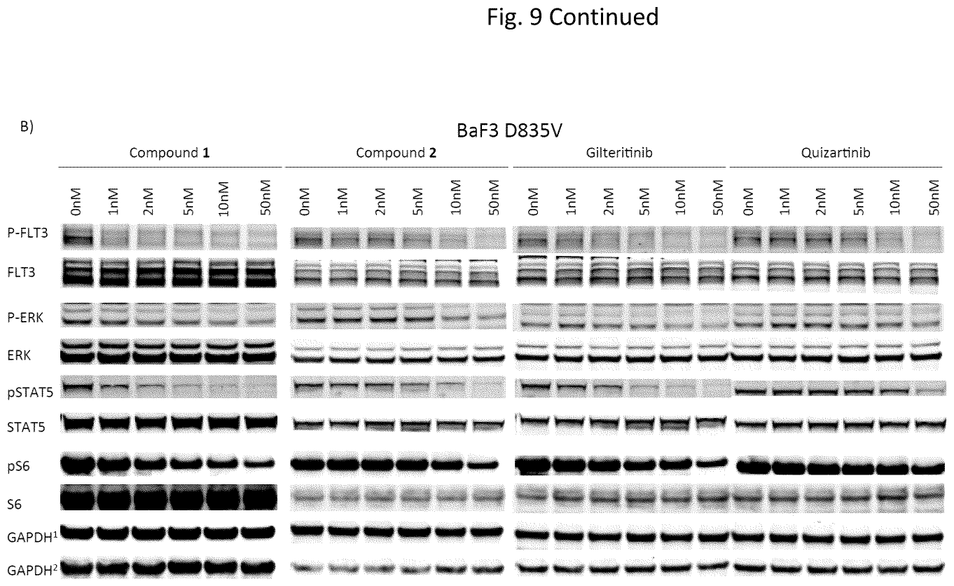

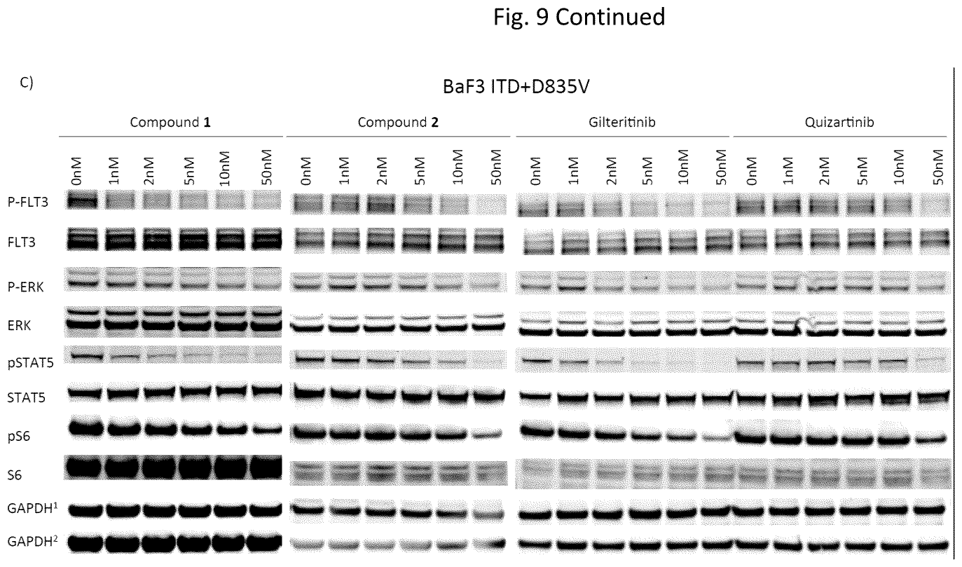

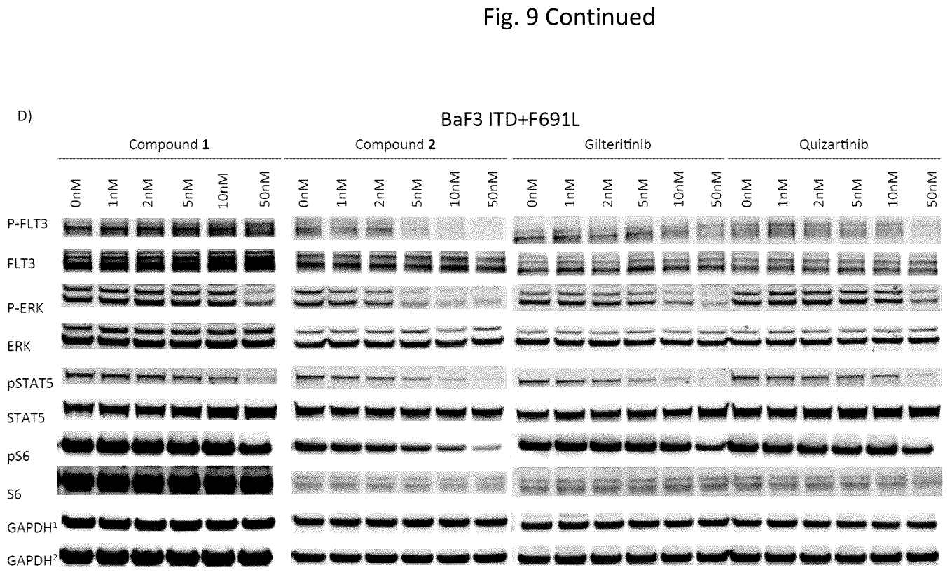

Compounds 1 and 2 were identified through parallel medicinal chemistry campaigns, starting from unique “hits” in a kinase-directed fragment screening library. Following multiple rounds of optimization from the two initial hits, a small series for each scaffold was assessed in cellular studies to select lead compounds. Cellular viability assays compared the activity of 1 and 2 to quizartinib and gilteritinib (FIG. 1A). Consistent with previous reports, quizartinib demonstrated exquisite potency against FLT3-ITD-expressing MV-4-11 and Molm14 cells, but severely diminished activity in Molm14 subclones harboring secondary resistance-conferring D835Y and F691L mutations. Gilteritinib demonstrated weaker activity against the parental FLT3-ITD-expressing lines, but as a type I inhibitor, retained activity against the D835Y mutant-expressing line. However, gilteritinib displayed less activity against the F691L mutation, consistent with the detection of this mutation in a subset of patients with loss of response to this agent. Both quizartinib and gilteritinib displayed little activity on the non-FLT3 driven HL60 and K562 lines. Compounds 1 and 2 both displayed activity comparable to quizartinib and superior to gilteritinib in MV-4-11 and Molm14 cells. Moreover, both retain activity against secondary resistant mutations D835Y and F691L at low concentration. Additionally, neither compound displayed any concerning toxicity against the non-FLT3 driven lines assessed. Similar results were obtained in Ba/F3 cells harboring various FLT3-ITD isoforms (FIG. 8).

The inhibition profiles were validated through evaluation of phospho-FLT3 and downstream signaling (FIG. 1B, C and FIG. S2). For both compound 1 (FIG. 1B) and compound 2 (FIG. 1C), low nanomolar concentrations were sufficient to inhibit phosphorylation of FLT3 and nearly abolish all downstream signaling in parental Molm14 cells. Compound 1, a presumptive type I inhibitor, displayed potent inhibition of phosphorylation in Molm14 D835Y, and although a decreased level of activity against Molm14 F691L was observed, this was comparable to gilteritinib and superior to quizartinib. Compound 2, a presumptive type II inhibitor, showed somewhat decreased ability to inhibit phosphorylation of FLT3 and downstream targets in Molm14 D835Y cells. However, compound 2 remained superior to quizartinib and fully ablated FLT3 phosphorylation at 50 nM concentration, similar to gilteritinib. In Molm14 F691L cells, Compound 2 was the most potent of all the inhibitors evaluated with a noticeable decrease in phosphorylation at 10 nM concentration and complete elimination at 50 nM. Similar results were obtained in Ba/F3 cells (FIG. S2).

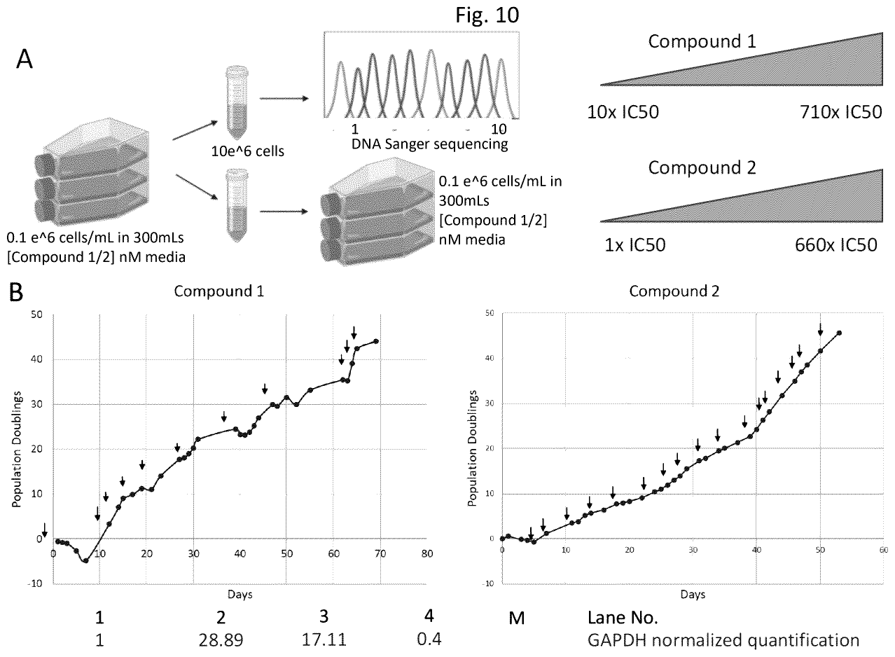

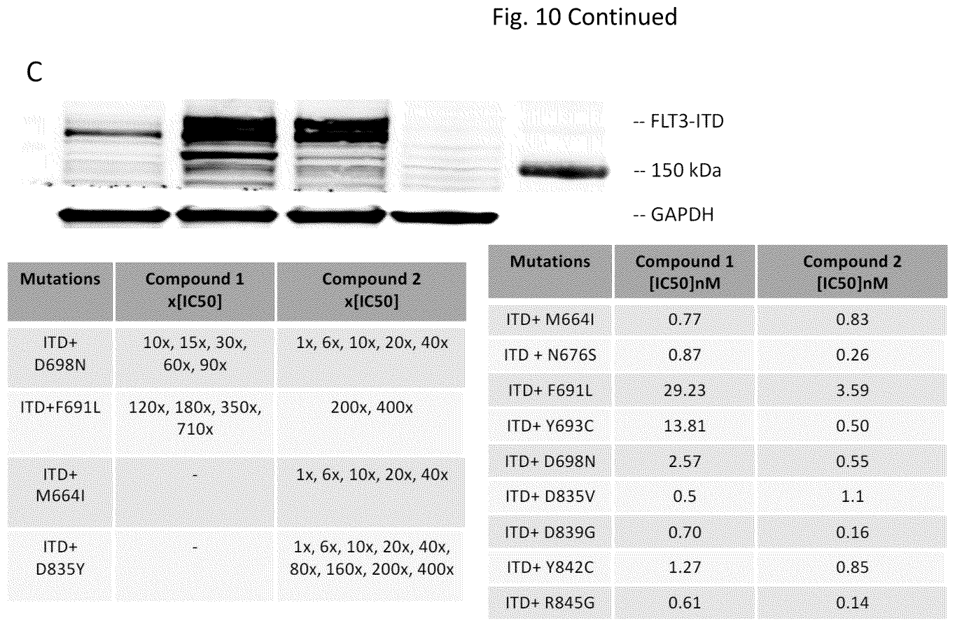

To determine if these compounds are potentially vulnerable to novel secondary mutations in FLT3-ITD, Ba/F3 cells were transduced with retrovirus generated using MSCVpuro/FLT3-ITD plasmid purified from the XL1-Red bacterial strain, which is deficient in DNA mismatch repair and introduces random point mutations. This assay has successfully identified point mutations for other FLT3 TKIs that were subsequently validated in samples obtained from patients with acquired resistance. Transduced cells were propagated in gradually increasing concentrations of compound 1 or compound 2. Kinase domain sequencing revealed a limited number of mutations detectable in cells that had acquired resistance. Of note, none of these conferred a greater degree of resistance than the F691L mutation when independently reintroduced into Ba/F3 cells. While it was possible to ultimately select for cells with considerable resistance to both compounds, Western immunoblot demonstrated the cause of resistance to be primarily a consequence of dramatically increased levels of protein carrying secondary kinase domain mutations. We further assessed several secondary mutations that confer relative resistance to other FLT3 TKIs, but none of these were capable of conferring significant resistance to compounds 1 and 2 (FIG. 10).

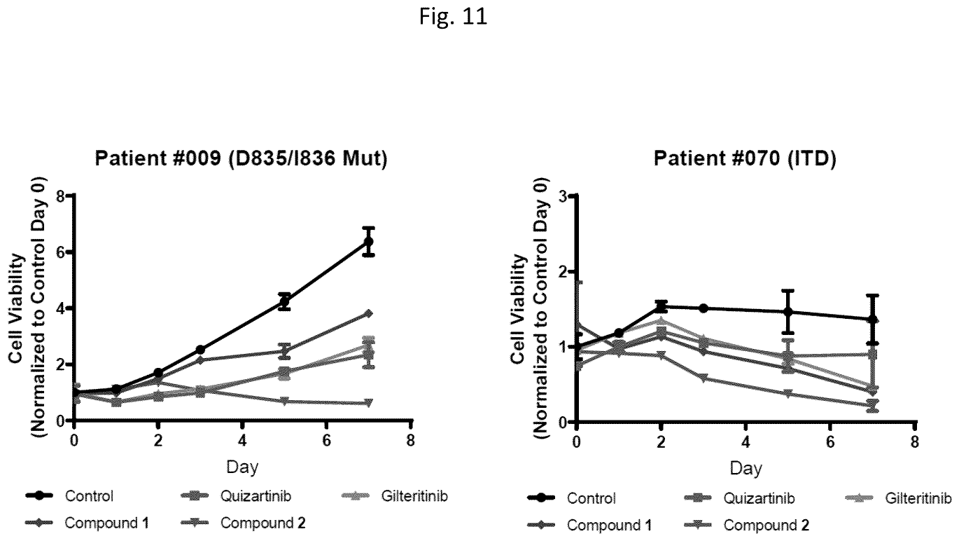

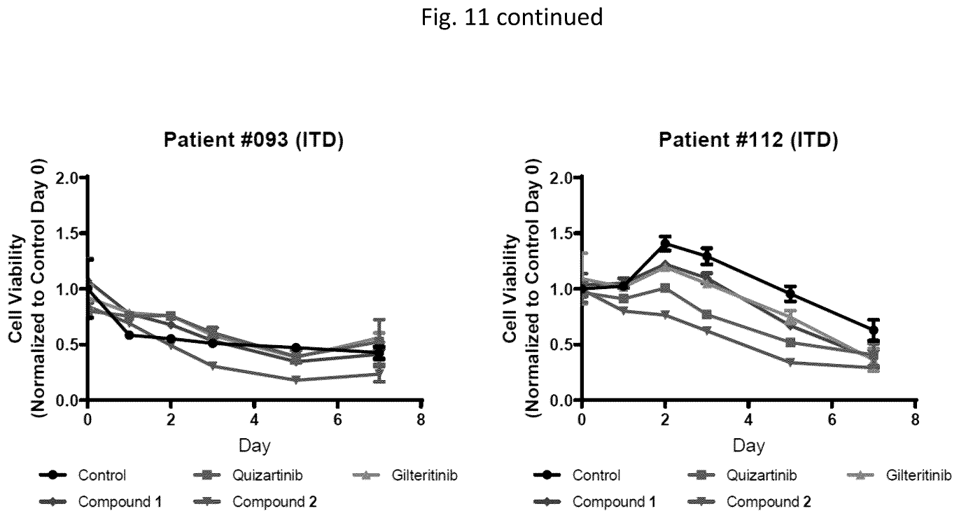

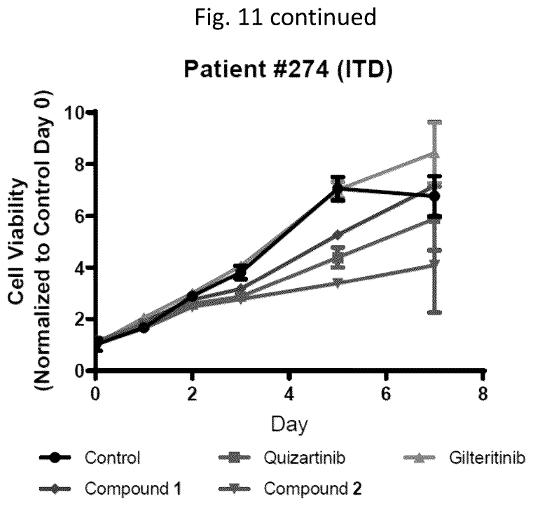

The compounds were further evaluated for activity against TKI-naïve primary AML patient samples expressing FLT3-ITD in short-term liquid culture growth inhibition assays (24). Compound 1 maintained activity comparable to quizartinib and gilteritinib in all samples while compound 2 displayed the most potent activity of all the compounds (FIG. 11).

A pitfall of some prior FLT3 inhibitors has been poor pharmacokinetic profiles. The plasma inhibitory assay identifies one pharmacokinetic profile by evaluating the activity of drugs in the presence of human plasma proteins that can sequester inhibitors in clinical experience. Compounds 1 and 2 displayed more potent inhibition of FLT3 in the presence of plasma compared to quizartinib and gilteritinib at the same concentrations. The presence of plasma had almost no impact on the activity of compound 1 (FIG. 1D).

Compound 1 and Compound 2 Display Less Toxicity than Gilteritinib Toward Normal Hematopoiesis

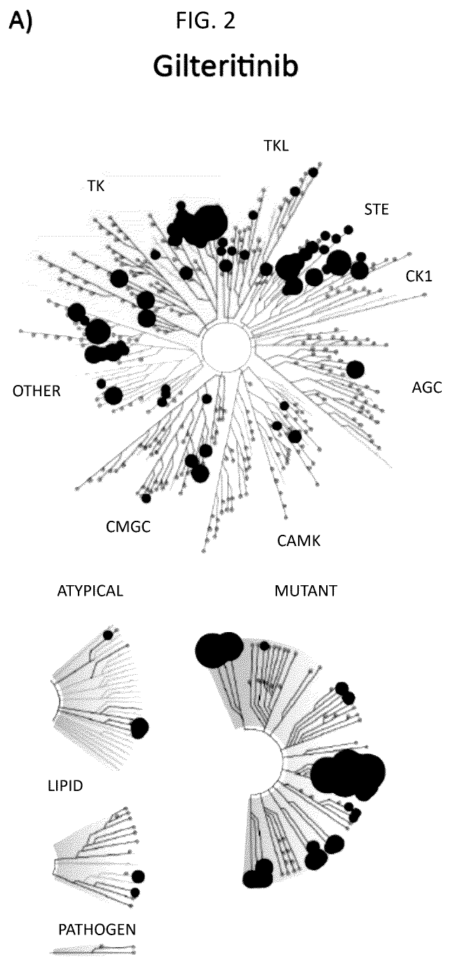

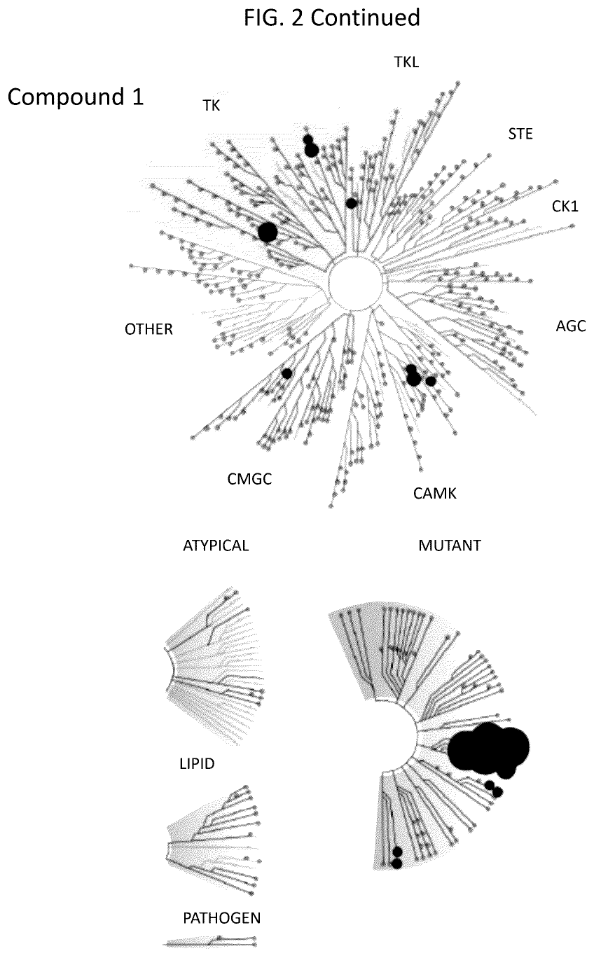

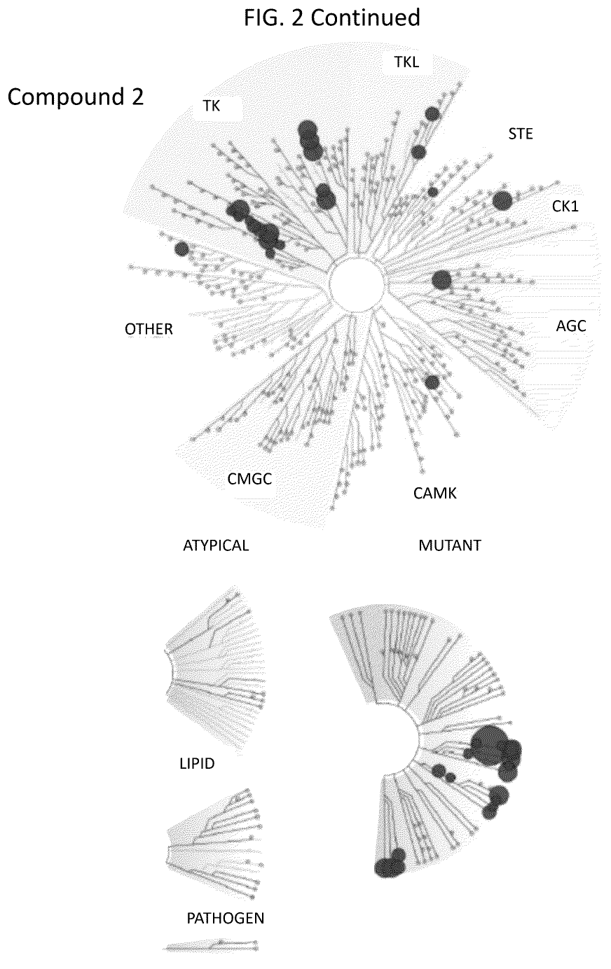

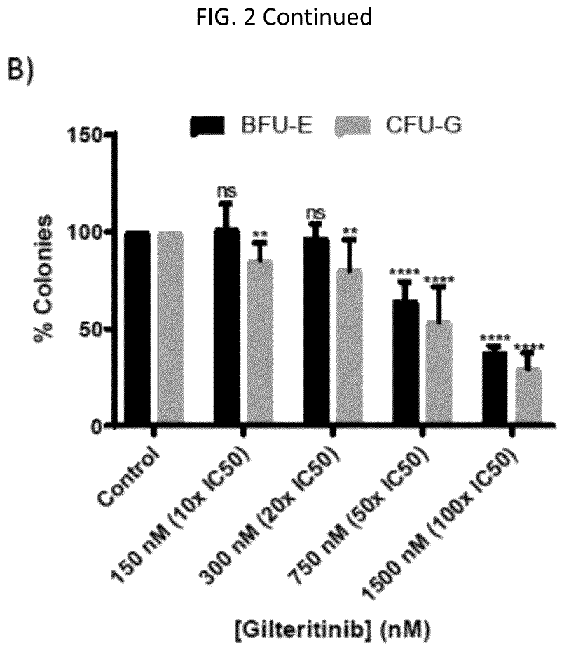

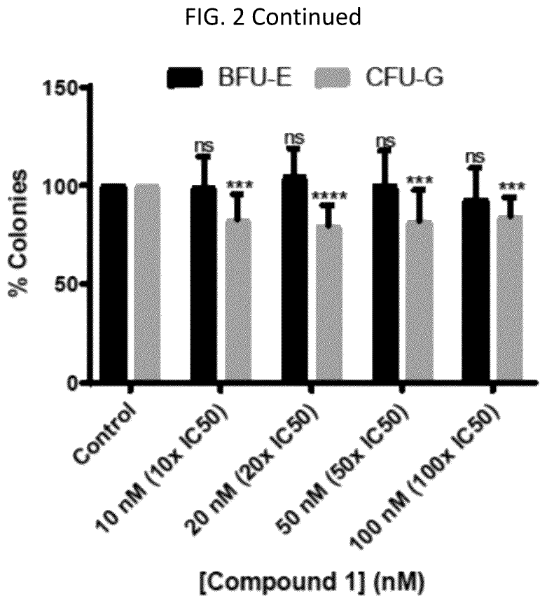

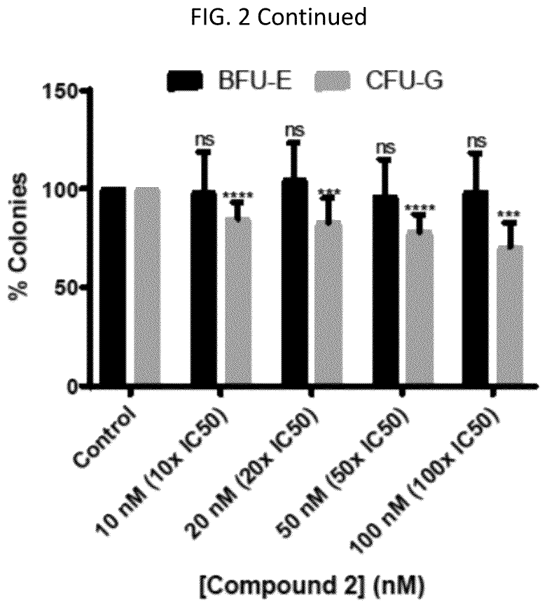

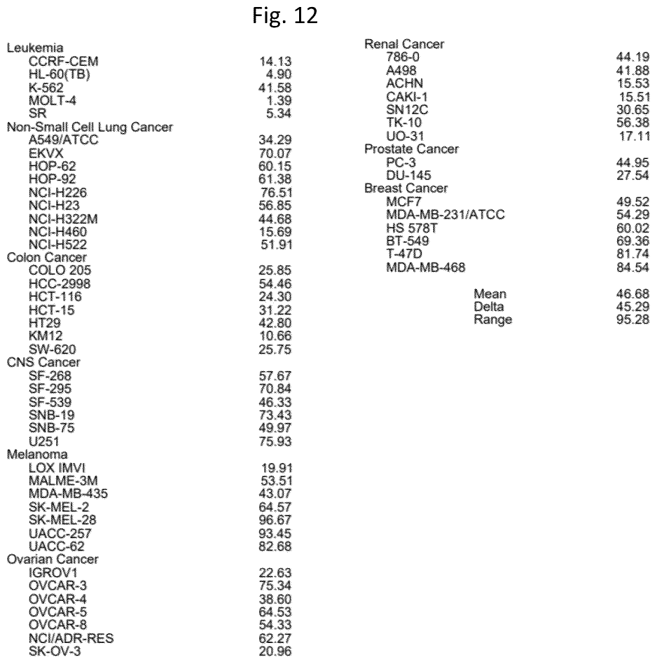

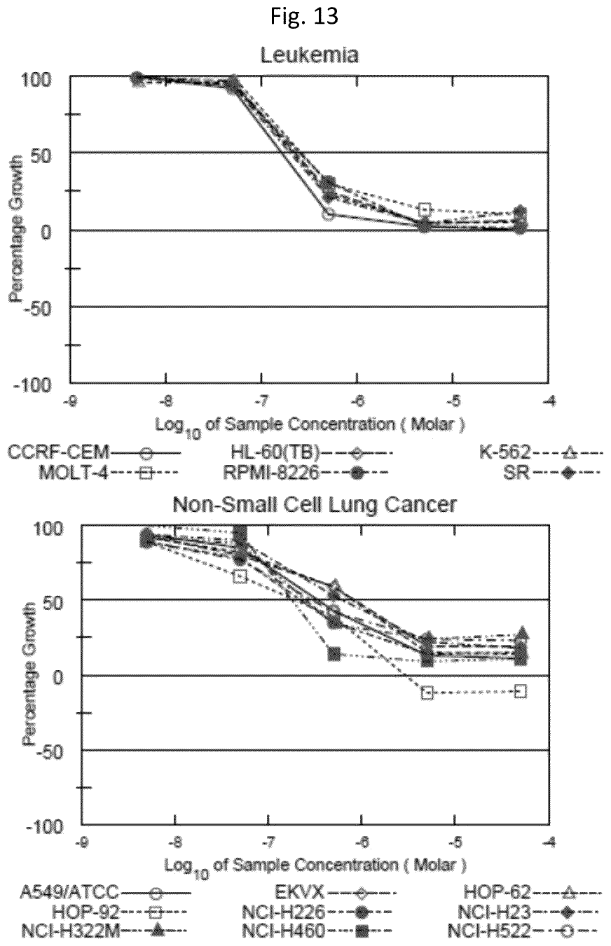

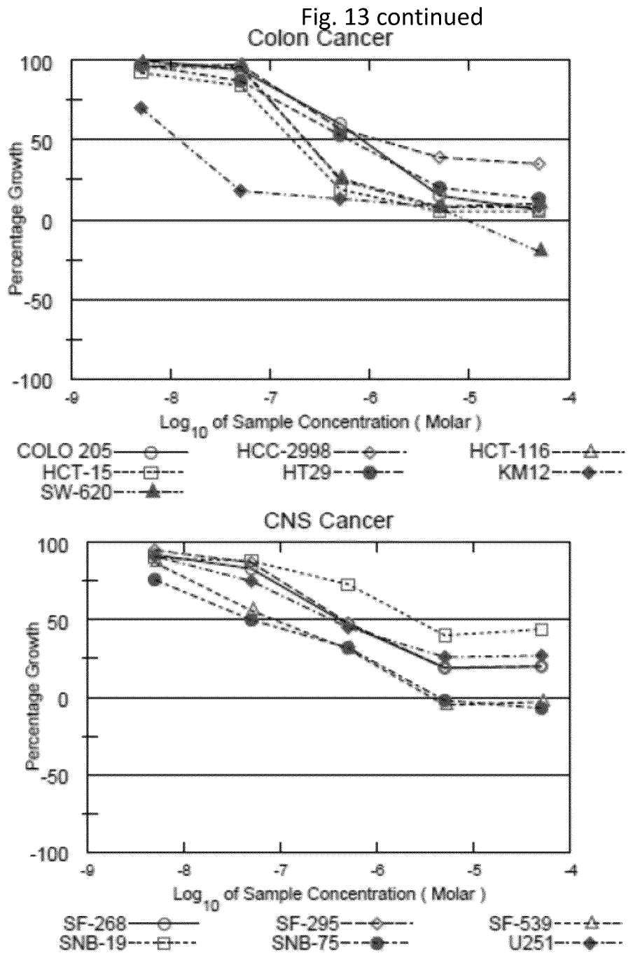

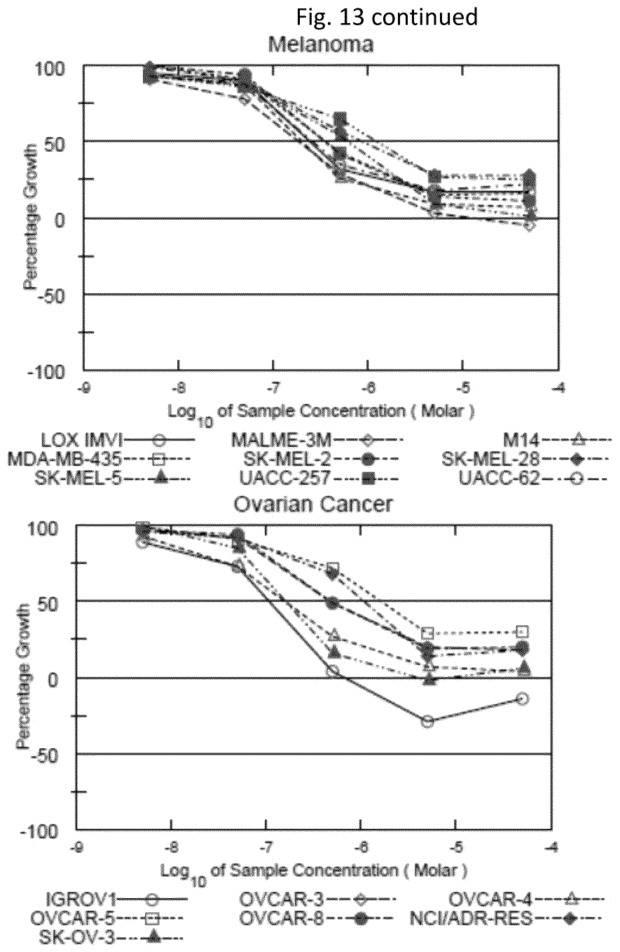

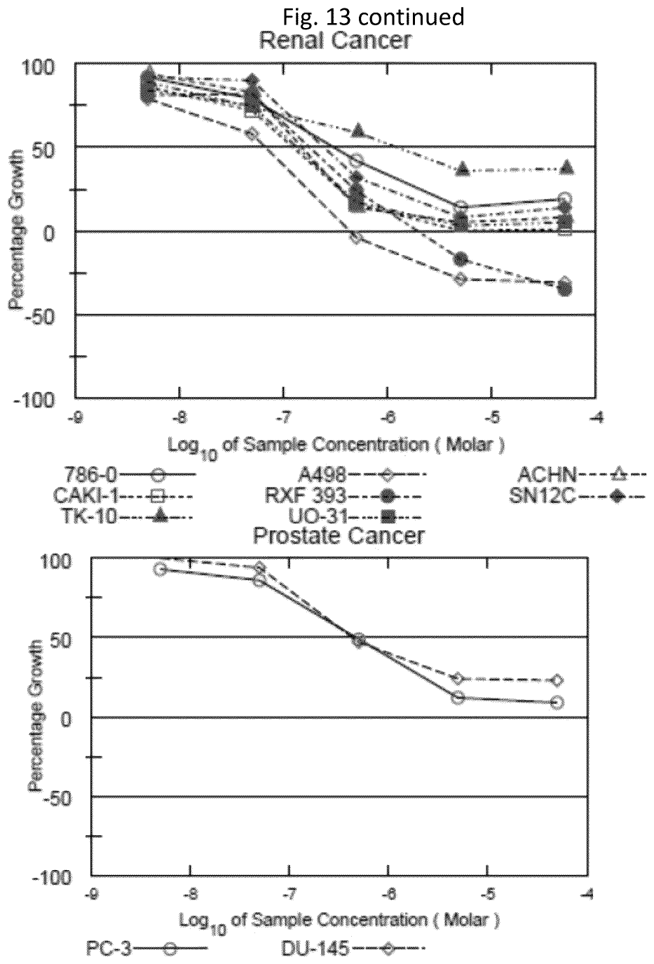

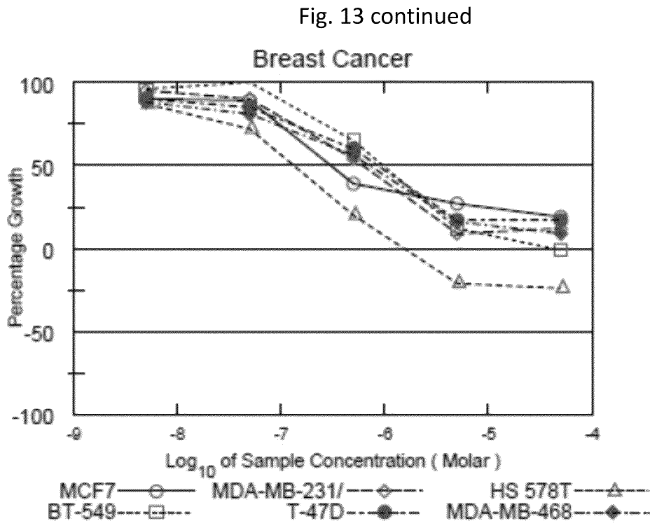

Myelosuppression represents a critical limitation of current FLT3 TKIs. To assess the selectivity profiles, KINOMEscan screening was performed for 1, 2, and gilteritinib at 20× the Molm14 IC₅₀ concentration of each compound (FIG. 2A). Compounds 1 and 2 bound to far fewer kinases than gilteritinib. Notably, compound 1 displays a remarkable selectivity profile. Further, neither compound displayed any significant cytototoxic activity in the NCI-60 cell line panel, which lacks cell lines that express mutant FLT3, although antiproliferative effects were noted at higher concentrations (FIGS. 12 and 13).

To determine if the improved selectivity of compounds 1 and 2 translated to less suppression of normal human hematopoiesis in vitro, we performed a colony forming unit (CFU) assay with normal donor bone marrow mononuclear cells (BM-MNC) (FIG. 2B). This assay is highly predictive of clinical myelosuppression. We compared compounds 1 and 2 to gilteritinib at equivalent multiples of their determined IC₅₀ values against Molm14 in the cell viability assay (FIG. 1A) in an effort to estimate the therapeutic window of each TKI. The combined results across three independent healthy donor samples demonstrated that when compared at equivalent doses, gilteritinib resulted in substantial impairment in BFU-E and CFU-G counts at 50× and 100×IC₅₀. In contrast, both compounds 1 and 2 had no significant impact on BFU-E growth at any concentration, and less impact on CFU-G growth than gilteritinib.

Molecular Docking Studies Suggest that Compound 1 Obtains Selectivity Through Mimicking an Aromatic Cage Interaction

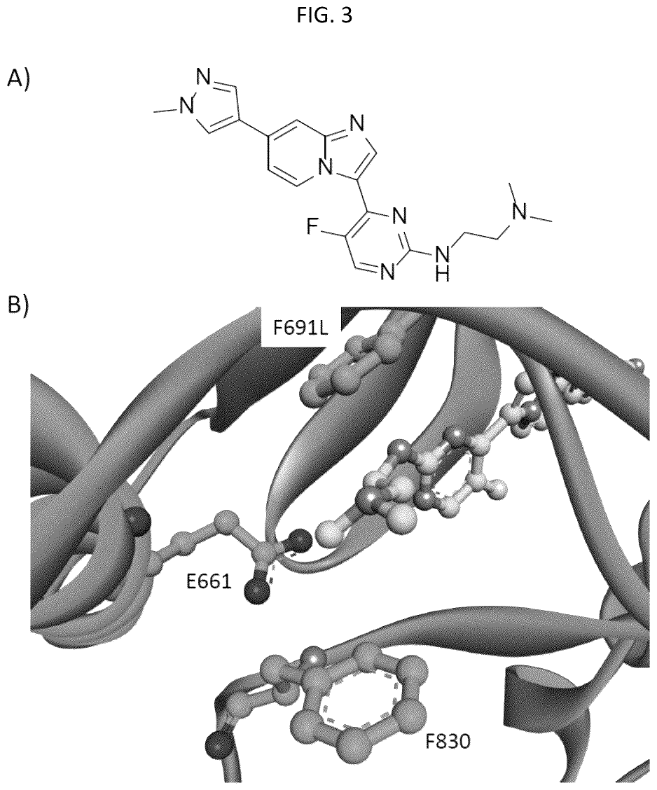

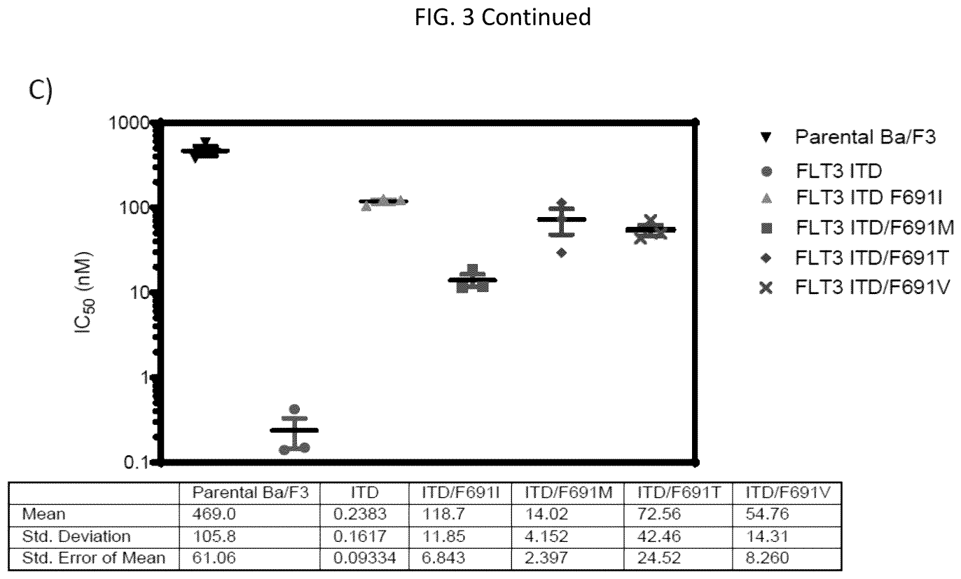

The high selectivity of compound 1 (FIG. 3A) suggests that compound 1 harbors unique binding features to achieve affinity toward FLT3. Molecular docking was employed to understand potential binding modes and their role in the selectivity of compound 1 (FIG. 3B). To observe compound 1 binding in the expected “DFG-in” conformation, a previously created FLT3 homology model created using a crystal structure of the closely related KIT kinase in its active conformation was utilized for docking studies (25). Interestingly, these studies revealed the dimethyl amine portion of compound 1 in close proximity to three key residues: the gatekeeper F691, as well as E661 and F830, in a manner that appears to mimic an “aromatic cage” interaction, a common biological mechanism for protein interactions, especially for the recognition of methyllysines by epigenetic readers (26,27). Aromatic cages are comprised of two to four aromatic side chains, and often incorporate an additional acidic side chain residue. Based on the modeling, the dimethylamine of compound 1 can form a salt bridge with E661 that is further stabilized by the F691 and F830 aromatic residues in an arrangement that mirrors a methyllysine within an aromatic cage. It is hypothesized this unique interaction plays a major role in the selectivity of compound 1. While E661 and F830 are conserved across the kinome, the phenyalanine residue in the gatekeeper position is present in only 14% of the kinome and is the only aromatic side chain of the 7 predominant gatekeeper residues comprising 97% of the kinome (28). This relative scarcity of phenylalanine in the gatekeeper position allows for inherent selectivity across a vast majority of the kinome. Notably, of the six kinases bound with greatest affinity in the KINOMEscan analysis (FLT3, IRAK4, PHGK1, PHGK2, CDK7, TRKA), with the exception of IRAK4, which contains a highly unusual tyrosine at this position, all have phenylalanine at the gatekeeper position.

To further investigate the possibility of a hydrophobic cage interaction and its contribution to the selectivity of compound 1, we assessed the impact of substitutions of the native phenylalanine at position 691 with the other residues most commonly found at this position in the human kinome (methionine, threonine, isoleucine, or valine). Ba/F3 cells transformed with FLT3-ITD or with FLT3-ITD substitutions at position 691 were assessed for sensitivity to compound 1 (FIG. 3C). Results showed a dramatic difference in IC₅₀ values between Phe gatekeeper FLT3-ITD and the FLT3-ITD with Met, Thr, Ile, or Val gatekeeper residues, with a nearly 100-fold loss of activity of the Ba/F3 FLT3-ITD F691M line relative to the Ba/F3 FLT3-ITD line, and an even greater impact on the other mutants.

### Key Molecular Feature Contributes to Improved Inhibition Profile of Compound 2

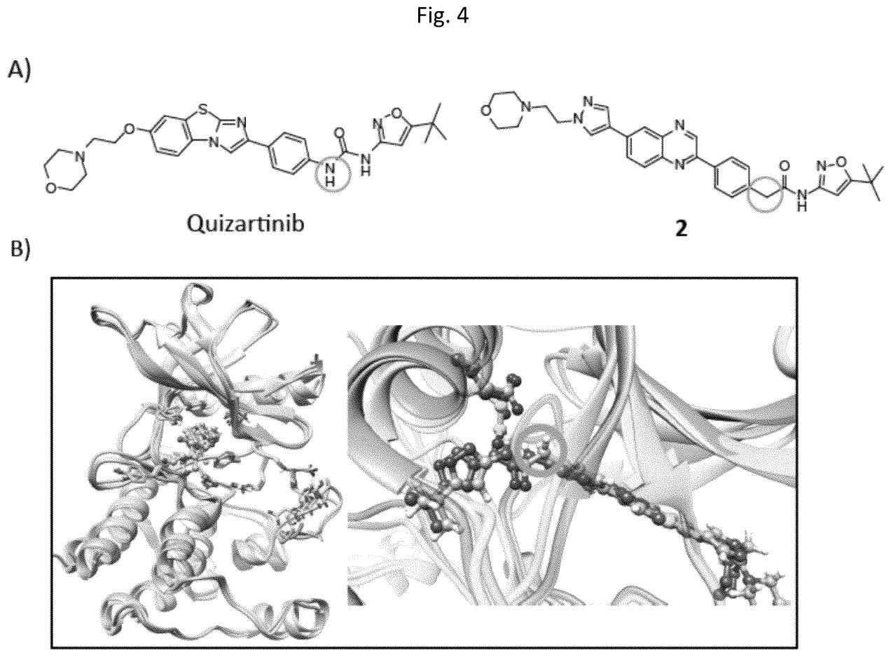

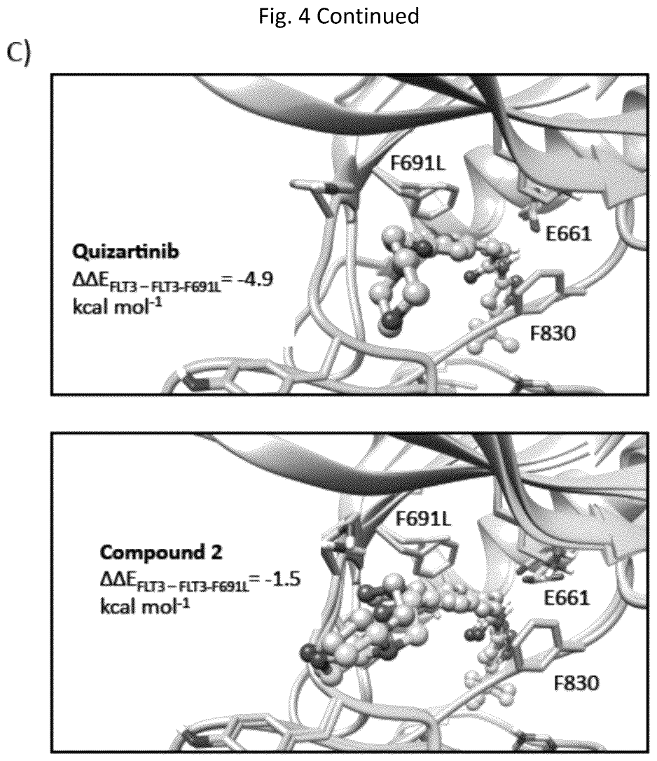

Compound 2 unexpectedly retains activity against substitutions at D835 and F691 that confer a high degree of resistance to quizartinib. Computational modeling was employed to further investigate this finding. There is only one difference within the allosteric binding portion between compound 2 and quizartinib: the linker of quizartinib is comprised of a urea functional group, while the linker of compound 2 is comprised of a methyl amide functional group (FIG. 4A). We tested whether this alteration might impact binding interactions within the FLT3 kinase domain.

We first modeled compound 2 within the inactive state crystal structure of FLT3 (FIG. 4B). As expected, compound 2 binds nearly identically to quizartinib in this conformation and forms a hydrogen bond with Glu661. The D835 mutation stabilizes an active kinase conformation of FLT3. When overlaying an active state model of FLT3 with the two inactive state models, the primary change is a conformational shift of the C-helix, resulting in a rotation of Glu661 into the allosteric pocket. This rotational shift places Glu661 directly in the space occupied by both inhibitors, causing obvious steric hindrance for both inhibitors. We hypothesized that compound 2 overcomes this steric hinderance due to the additional degree of freedom within the carbon bond compared to the rigid urea linker of quizartinib. The extra freedom likely allows for 2 to accommodate different binding modes with minimal impact to its overall affinity, whereas quizartinib is unable to accommodate the conformational shift without much more dramatic alterations of its binding mode.

To evaluate the F691L mutation, we calculated the relative binding energies of both quizartinib and compound 2 in the native FLT3 binding pocket, and in a model of a binding pocket with the F691L mutation, using MM/GBSA calculations (FIG. 4C). For quizartinib, the calculated free energy changes of binding in native FLT3 and in the F691L mutant were-105.3 kcal/mol and −100.4 kcal/mol respectively, meaning there was nearly a 5 kcal/mol difference between the two binding energies. For compound 2, the calculated free energy changes were −104.6 kcal/mol for the native and −103.1 kcal/mol for the F691L mutation, resulting in only a 1.5 kcal/mol shift in binding affinity. We hypothesized the larger difference in binding energies for quizartinib was due to two reasons: i) increased dependence on pi-pi interactions between the F691 gatekeeper residue and the pi resonance system present throughout quizartinib from the phenyl group, through the urea linker and into the isoxazole group, and ii) the rigidity of quizartinib prevents alternative binding positions to accommodate secondary mutations. Conversely, because compound 2 has less resonance through its chemical structure due to the methyl amide linker, it has less dependency on this key interaction. Further, the carbon in 2 contributes to a higher lipophilicity and flexibility in the local region enabling binding interactions to establish a local energy minima with the leucine containing mutant. The urea linker of quizartinib is less lipophilic and predicted to be unable to recoup any lost interactions in the presence of leucine. This is evidenced by the relative change in binding modes adopted by quizartinib and compound 2 in the wildtype FLT3 pocket versus the F691L FLT3 pocket (FIG. 4C). Quizartinib has a nearly identical binding mode in both models, suggesting it is incapable of altering its position or engaging in new contacts with the protein. Conversely, compound 2 alters its positioning within the pocket to accommodate the F691L mutation and better maintain affinity.

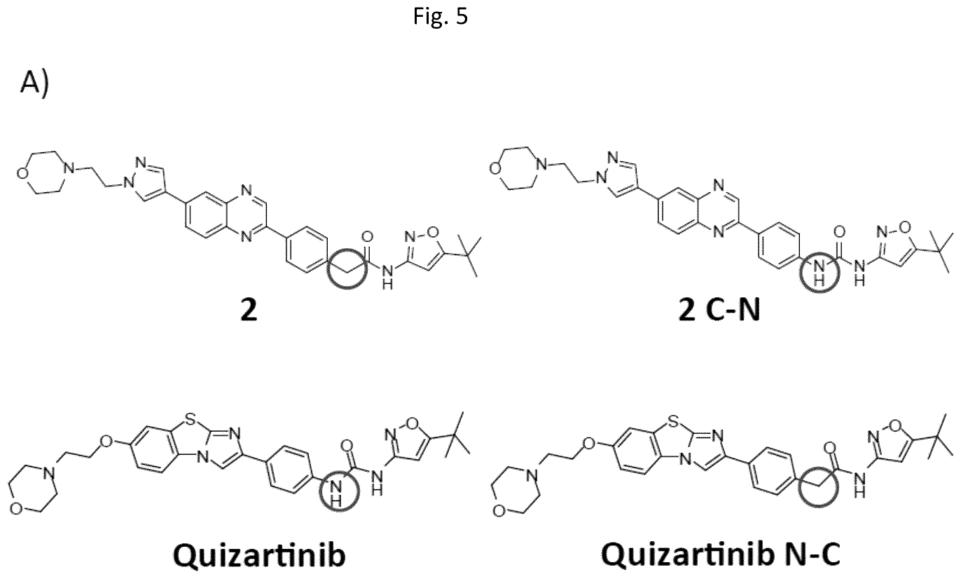

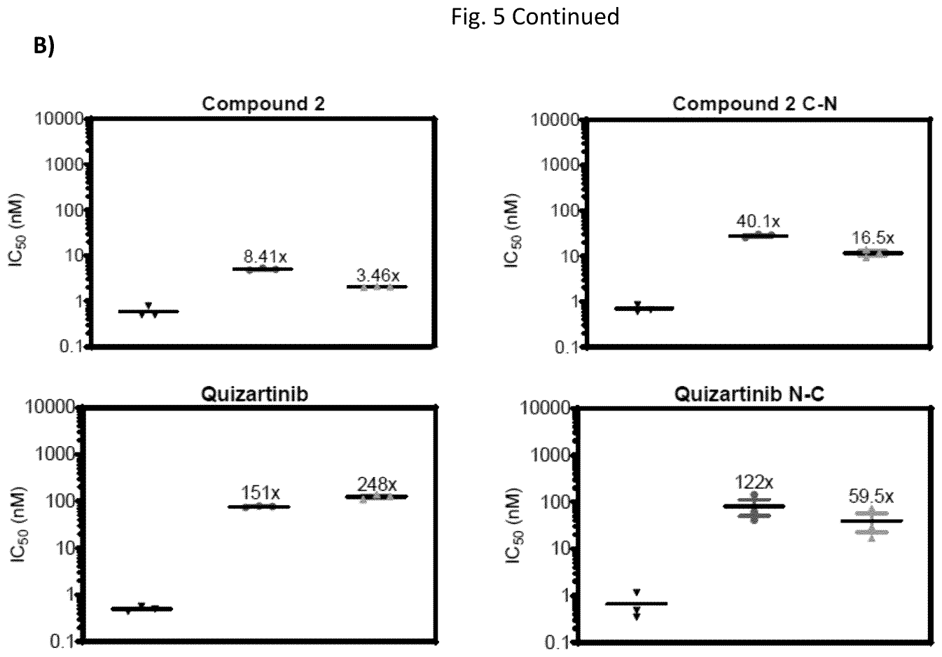

To test these hypotheses, we synthesized analogs of compound 2 and quizartinib (FIG. 5A). The compound 2 analog (2 C—N) has a carbon to nitrogen substitution creating a urea linker matching the structure of quizartinib. The quizartinib analog (quizartinib N—C) has a nitrogen to carbon substitution to create a methyl amide linker matching compound 2. We hypothesized that 2 C—N would have a greater disparity compared to 2 between its activity against the FLT3 ITD mutation vs either the D835Y or F691L mutations, and quizartinib N—C would have less of a disparity compared to quizartinib between FLT3-ITD and the secondary mutants.

Notably, the activities of both analogs against the Molm14 cell line were nearly identical to that of their respective parental molecules (FIG. 5B). However, compound 2 C—N was substantially less active against both the D835Y and F691L mutants, with a relatively 5-fold increase in both the D835Y: ITD and F691L: ITD IC₅₀ ratios compared to compound 2. The activity of quizartinib N—C also supported the hypothesis, albeit to a lesser extent. The D835Y: ITD IC₅₀ ratio of the analog was modestly improved compared to that of quizartinib. However, there was a 4-fold decrease in the margin between the F691L: ITD IC₅₀ ratios for the analog and parental molecule respectively.

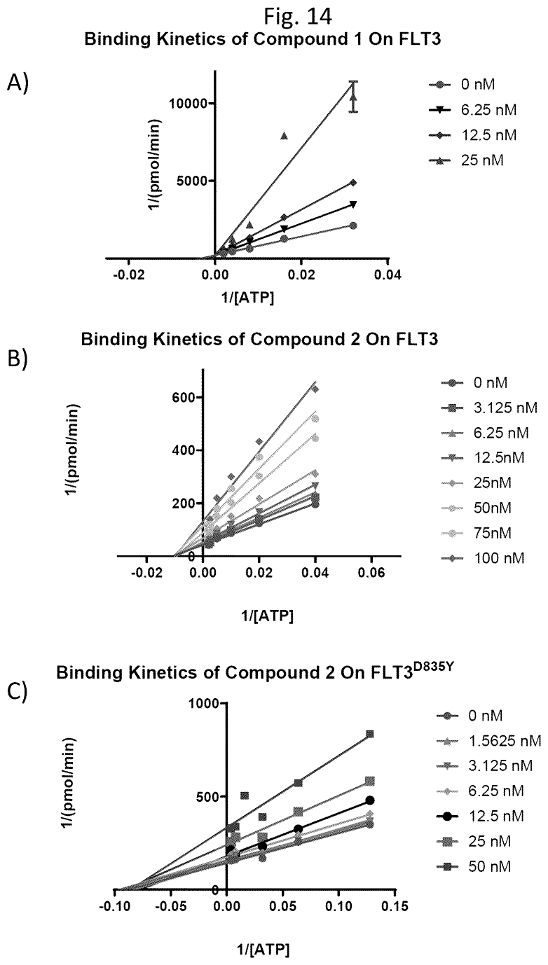

To further assess putative binding modes of compounds 1 and 2, enzyme kinetic analyses were performed. As predicted by molecular docking studies, analysis of compound 1 with FLT3 supports a type 1 binding mode, whereas studies of compound 2 support a type 2 binding mode. Interestingly, analysis of compound 2 with FLT3 D835Y supports an intermediate conformation between type 1 and type 2, consistent with the molecular docking studies (FIG. 14).

### Compound 1 and Compound 2 Display Activity In Vivo and Compound 2 Outperforms Gilteritinib in F691L Resistance Model

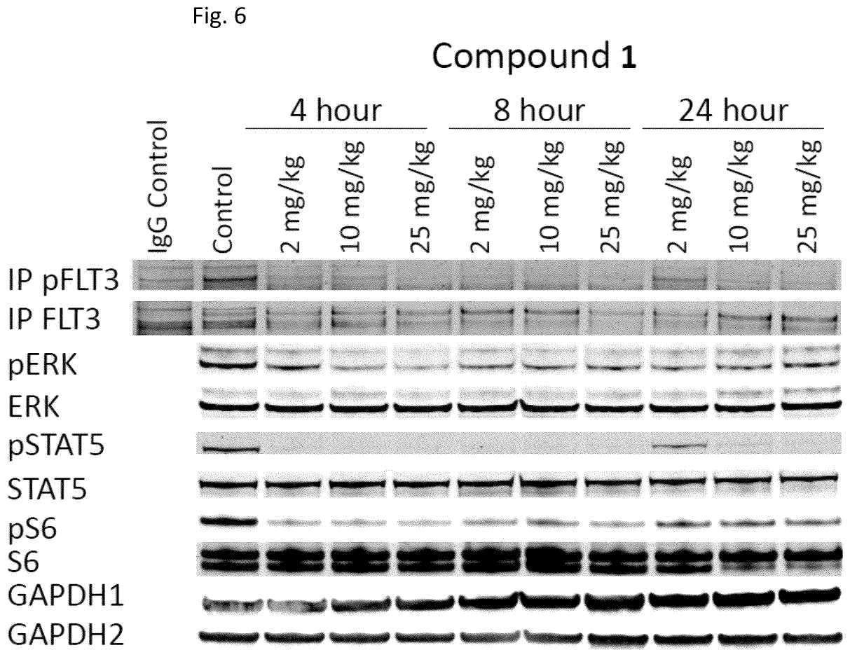

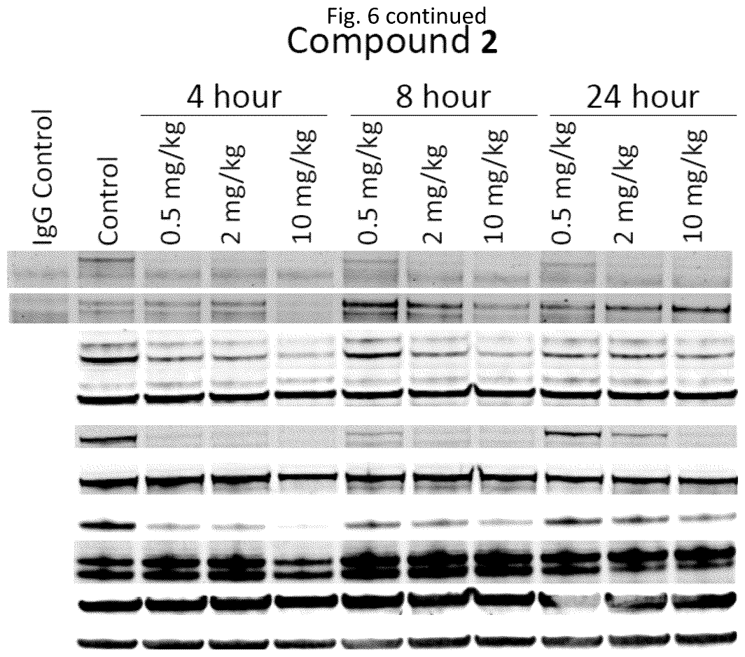

To assess if compounds 1 and 2 possess drug-like qualities, we first evaluated Molm14 subcutaneous xenografts to confirm target inhibition and examine the duration of response following a single dose of each compound by oral gavage. (FIG. 6). Both compounds displayed inhibition of target and downstream effectors at all doses at 4 hour and 8 hours. For compound 1, both the 2 mg/kg and 10 mg/kg doses were sufficient for retaining target inhibition for 24 hours. For compound 2, kinase signaling was restored at 24 hours following treatment with the lower doses, but the 10 mg/kg dose achieved sustained target inhibition. These studies demonstrated oral bioavailability and acceptable pharmacokinetic profiles for future studies to achieve in vivo target inhibition of FLT3-ITD.

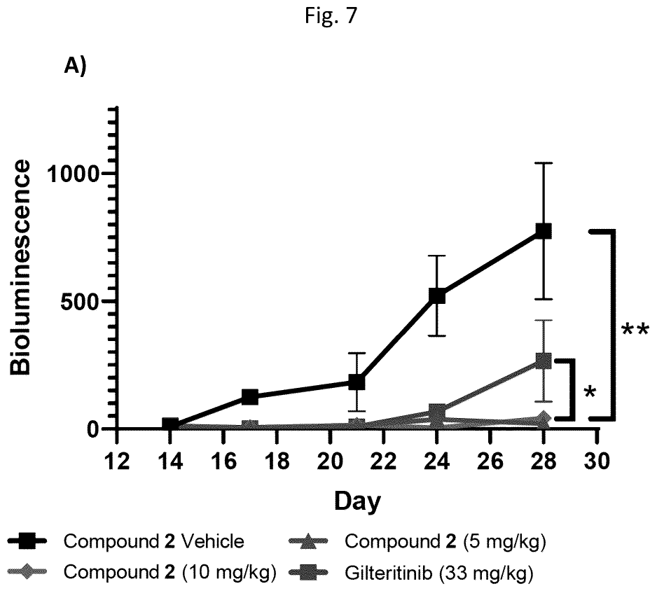

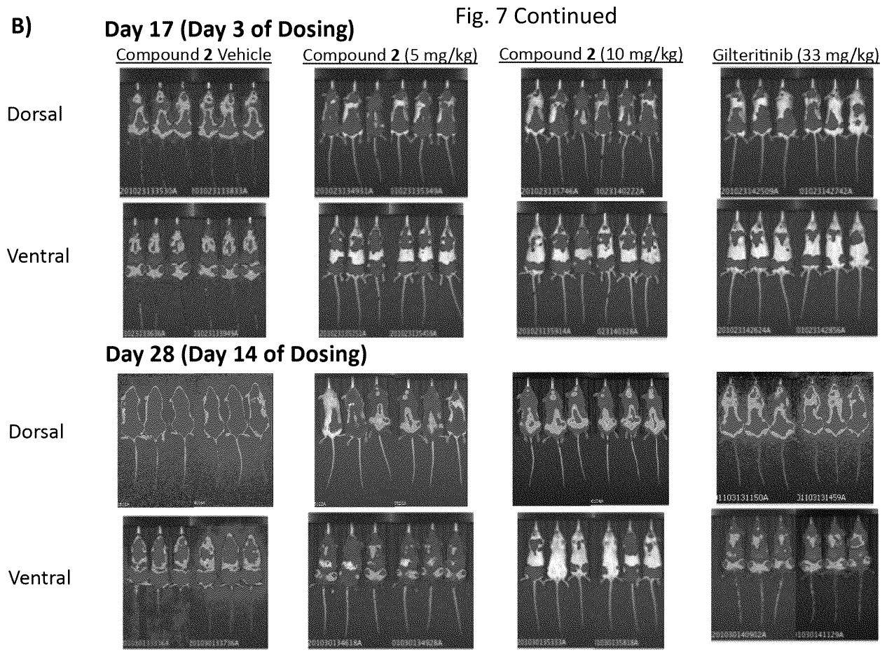

A circulating tumor model was used to compare compound 2 and gilteritinib on their ability to prevent tumor growth. Given the success of gilteritinib in treating FLT3-ITD and FLT3 D835 secondary mutations, the F691L mutation was employed. Molm-14 F691L cells stably expressing firefly luciferase were injected into the tail vein of NSG mice and tumor growth was monitored through bioluminescence. Mice were dosed with compound 2 (5 or 10 mg/kg), gilteritinib (33 mg/kg=maximally tolerated dose), or vehicle control. Both gilteritinib and compound 2 were able to slow tumor growth compared to control, however both doses of compound 2 were able to nearly halt all tumor growth and demonstrated a statistically significant difference compared to gilteritinib (FIG. 7). The promising activity of compound 2 against the F691L secondary mutation in this study demonstrates its promise for effectively preventing clinical acquired resistance due to this resistance mechanism. Efforts are ongoing to optimize a formulation for administration of compound 1 in this model system.

### Discussion

The emergence of target therapy, particularly the development of potent and selective kinase inhibitors, has changed the treatment landscape across multiple tumor types. BCR-ABL1 inhibitors have transformed Philadelphia chromosome-positive (Ph+) chronic myeloid leukemia (CML) into a manageable chronic condition, with life expectancies approaching that of the general population (29). This achievement was possible through comprehensive studies to understand the molecular mechanisms of resistance to early inhibitors and subsequent development of next generation inhibitors invulnerable to such resistance developments (30-35). However, patients with the most advanced stage of CML (“blast-crisis” CML), as well as patients with pediatric Ph+ ALL, suffer from low response rates to BCR-ABL TKI monotherapy. Encouragingly, Ph+ ALL survival rates have dramatically improved through combinations of chemotherapy and TKI therapy, followed by TKI “maintenance” therapy for at least several years (36). It therefore is reasonable to hypothesize a similar treatment regimen that incorporates AML chemotherapy and potent, selective FLT3 TKI therapy could result in a substantially increased cure rate. A key feature of BCR-ABL1 inhibitors that enables their long-term use as a maintenance therapy is their lack of myelosuppressive activity. To date, no active FLT3 inhibitors display this feature. The impact of quizartinib and gilteritinib on normal hematopoiesis is well documented. FF-10101, a covalent FLT3 inhibitor currently in clinical trials, also demonstrates KIT inhibition, and its myelosuppressive properties in vitro have been documented (16). It is therefore crucial that next generation FLT3 inhibitors not only retain activity against all clinically relevant drug-resistant mutant isoforms of FLT3, but also limit myelosuppression.

Here we describe two chemically distinct novel potent and selective FLT3 TKIs with different modes of binding to FLT3 (DFG-in vs DFG-out), that display characteristics of potential “best in class” FLT3 TKIs. Compounds 1 and 2 demonstrated more comprehensive activity against FLT3-ITD and the secondary D835 and F691 FLT3 mutations than either quizartinib or gilteritinib. Additionally, we failed to identify any secondary kinase domain mutations that were capable of conferring significant resistance to these compounds. Broad selectivity screening through KINOMEscan and the NCI-60 panel demonstrated that both compounds were more selective than gilteritinib and largely non-cytotoxic against in the NCI-60 cell line panel. Ultimately, this selectivity resulted in an encouraging lack of suppression of normal human hematopoiesis in vitro when compared with concentrations of gilteritinib expected to achieve the same degree of FLT3 inhibition. Molecular docking studies identified key interactions within the FLT3 binding pocket that we further explored through both inhibitor analog creation and enzyme mutagenesis approaches. Docking studies elucidated a key selectivity feature within compound 1 through an interaction between the dimethyl amine of the inhibitor and the FLT3 binding site resembling the hydrophobic cage mechanism of epigenetic readers, and a key structural feature of compound 2, the methyl amide linker, that allows for maintaining efficacy against activation loop mutations.

Molecular docking and enzyme kinetic studies of both inhibitors provide unique insights to future TKI discovery campaigns. Outside of a select few circumstances, type I inhibitors have historically displayed less selectivity than other types of kinase inhibitors. To the best of our knowledge, utilizing an aliphatic amine chain to occupy the gatekeeper region of the active site is a relatively unexplored feature for TKIs. By possibly mimicking an evolutionally conserved biological interaction, the dimethyl amine feature of compound 1, a presumptive type I inhibitor, provides a potential unique tool for targeting select kinases. The de facto hydrophobic cage formed through the phenylalanine gatekeeper residue and conserved glutamate and phenylalanine residues is found in only a subset of kinases, and notably, KINOMEscan profiling shows compound 1 has affinity almost exclusively for kinases containing phenylalanine gatekeeper residues. The incorporation of the dimethyl amine then serves as an effective method to quickly gain selectivity toward kinases with phenylalanine or other aromatic gatekeeper residues. While the risk for mutations occurring with the gatekeeper residue is high, compound 1 nonetheless maintains efficacy against the clinically-relevant F691L mutant suggesting sufficient potency can still be achievable due to additional interactions within the molecule.

Prior type II TKIs (quizartinib, pexidartinib, imatinib, sorafenib, regorafenib) have been vulnerable to activation loop mutations across class III RTKs. However, compound 2, a type II inhibitor, remarkably maintains low nanomolar activity against FLT3 D835 mutant isoforms. The Examples demonstrate that type II inhibitors can overcome this resistance mechanism, and that simple alteration of the linker region of the molecule leading into the allosteric pocket may enable greater ability to tolerate mutations that favor an active kinase conformation. While rigidity is often a desirable feature within small molecule drug development, our investigation of linker analogs for compound 2 and quizartinib serves as an interesting counterexample in which the extra degree of freedom allows compound 2 to accommodate a shifting binding pocket. Notably, numerous type II inhibitors have implemented the urea functional group or other bulky additions that may hinder mobility at this position. Like quizartinib, both regorafinib and sorafenib contain a urea linker group and have demonstrated susceptibility to activation loop mutations in their kinase targets (37,38). Our findings suggest similar substitutions to the methyl amide linker may enable these drugs to be more tolerant of such substitutions.

In summary, we describe the creation and characterization of two novel FLT3 inhibitors with best-in-class properties. These compounds represent the first FLT3 TKIs to display a high degree of potency and selectivity toward FLT3-ITD, comparable or superior activity against clinically relevant FLT3 mutant isoforms compared with quizartinib and gilteritinib, and significantly reduced toxicity toward normal hematopoietic stem and progenitor cells in vitro. In addition, both compounds appear to be orally bioavailable in mice and capable of effecting durable target inhibition. Deeper analysis of the binding mechanisms of both compounds identified unique insights that serve to support future medicinal chemistry efforts. Confirmation of their predicted modes will require crystallographic studies, which are ongoing, as are studies required to move these compounds toward clinical application.

### Materials and Methods

### Inhibitors

Compounds 1, 2, 2 C—N, and quizartinib N—C were synthesized. Quizartinib was purchased from Selleckchem (Houston, TX). Gilteritinib was provided by Astellas Pharma (Martinez, CA).

The synthetic scheme for preparing compound 2 is provided below and is representative for the preparation of the compounds disclosed herein.

### Cell Lines

MV-4-11 and parental Molm-14 cells were a gift from Dr. Scott Kogan (University of California, San Francisco). HL60 and K562 cell lines were obtained from Charles Sawyers. Mutant Molm-14 lines were isolated through selection in the presence of quizartinib as previously described (18). Briefly, Molm-14 cells were cultured in increasing concentrations of quizartinib and subsequently subcloned. Sanger sequencing identified secondary FLT3 mutations. Stable Ba/F3 lines were generated by retroviral infection as previously described (5). All cell lines were tested to be mycoplasma free and human cell lines were authenticated through STR profiling completed at the University of California, Berkeley.

### Cell Viability Assay

Exponentially growing cells (1×10³-1×10⁴ cells) were plated in triplicate in a 96-well plate with 0.1 mL of RPMI 1640+10% (vol/vol) FBS. Each triplicate was either dosed with drug or DMSO vehicle control. Drug dosing was completed at log doses ranging from 0.1 nM-10 μM. Cell viability was assessed after 48 hours using CellTiter GLO (Promega). Viability values were normalized to the vehicle control average. Numerical IC₅₀ values were calculated in GraphPad Prism 7.

### Immunoblotting

Exponentially growing Molm-14 or Ba/F3 cells were plated with RPMI 1640+10% (vol/vol) FBS with the indicated amount of drug. Following 120 minute incubation at 37° C., cells were washed in PBS and lysed in HEPES lysis buffer supplemented with protease and phosphatase inhibitors and processed as previously described (39). Immunoblotting was performed using anti-phospho-tyrosine 4G10 antibody (Millipore Sigma) and anti-FLT3 antibody (Cell Signaling Technology) following immunoprecipitation with anti-FLT3 antibody from 500 μg of total protein. Immunoblotting of ERK, STAT5, S6 and GAPDH was performed with anti-phospho-ERK, anti-ERK, anti-phospho-STAT5, anti-STAT5, anti-phospho-S6, anti-S6 and anti-GAPDH antibody (Cell Signaling Technology)

### Plasma Inhibitory Assay

Plasma Inhibitory Assay was performed as previously described with modifications (40). Ba/F3 cells (7×10⁶ cells) stably expressing FLT3-ITD were resuspended in 1 mL of RPMI 1640+10% FBS or human plasma from a healthy control. All samples were collected under the University of California, San Francisco institutional review board (IRB)-approved cell banking protocol (CC #112514). Informed consent was obtained in accordance with the Declaration of Helsinki. The appropriate amount of drug was added and incubated at 37° C. for 120 minutes. Following, cells were washed in PBS and lysed in HEPES lysis buffer supplemented with protease and phosphatase inhibitors and processed as previously described (40,41). Immunoblotting was performed using anti-phospho-FLT3 and anti-FLT3 antibody (Cell Signaling).

### Colony Forming Unit Assay of Normal Bone Marrow

Bone marrow mononuclear cells (BM-MNC) of healthy donors (n=3) were purchased from StemCell Technologies (Vancouver, Canada). Cells were plated in a 35 mm dish in triplicate with methylcellulose medium at a concentration previously determined to form approximately 100 total colonies, usually 30,000 cells/mL and containing the appropriate amount of drug. Dishes were incubated for 14-15 days and visually assessed for erythrocyte burst-forming units and granulocyte-macrophage colony-forming units.

### Computational Modeling

The crystal structure of the wild-type FLT3 kinase domain bound to quizartinib (PDB ID: 4XUF), which adopts an inactive conformation, was used as a reference for building models of wild type and mutant FLT3 bound to GN-QBA-16. These complexes were minimized by Embrace (Schrodinger®) followed by MM/GBSA calculations Prime (Schrodinger®) allowing flexibility of residues 5 Å from the ligands (42,43). The crystal structure of KIT kinase domain (PDB ID: 1PKG) was used as a reference for modeling the active conformation of wild type and mutant FLT3 bound to F371-037.2. Structural discussions were performed in virtual reality using the ChimeraX software (44,45).

### Generation of FLT3 Gatekeeper Mutants

Selected mutations were engineered into MSCVpuroFLT3-ITD through the QuickChange mutagenesis kit (Agilent) as previously described (5). Primers used are available in the Supplemental Materials.

### Synthesis of Compound 2

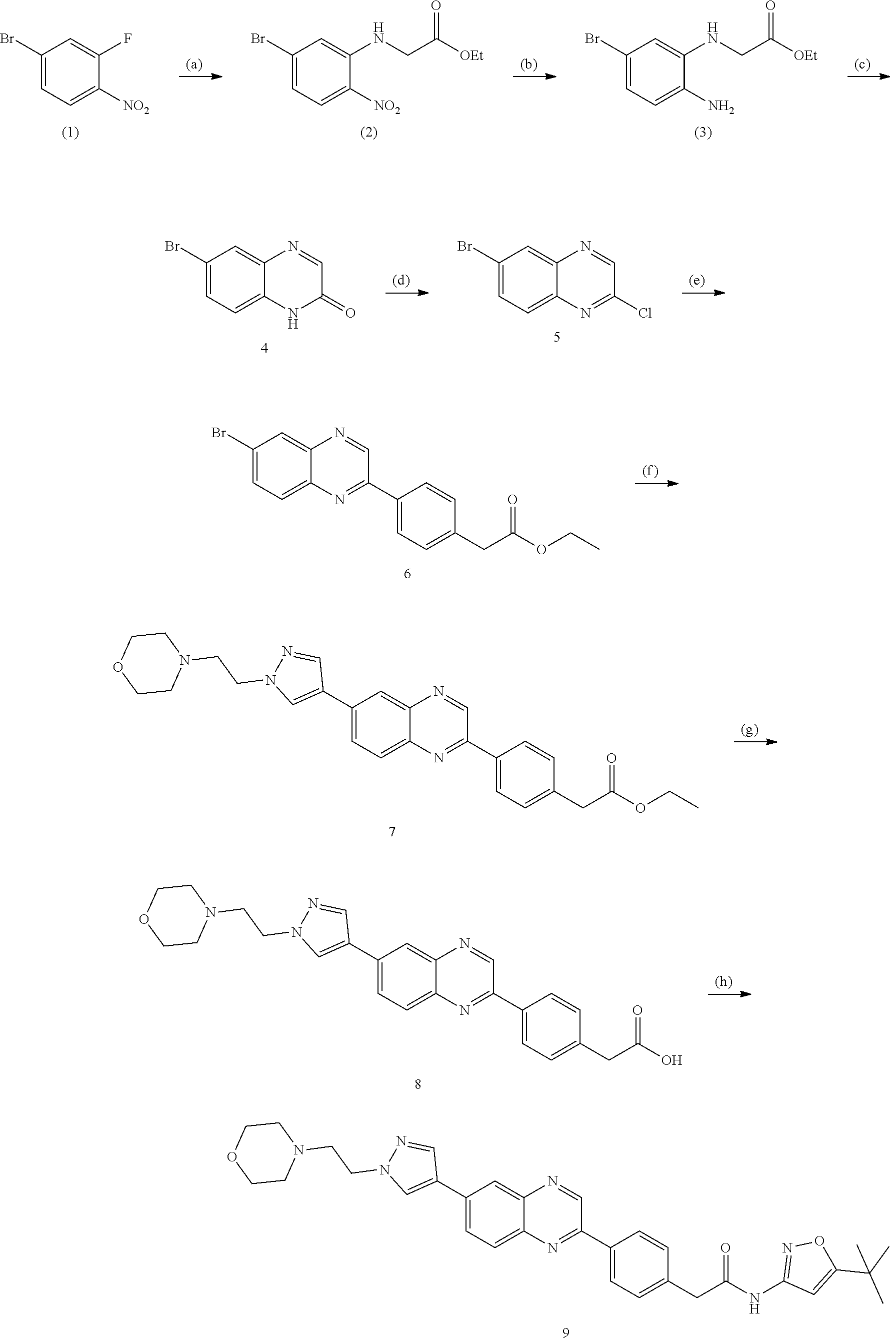

Reagents and conditions: (a) glycine ether ester hydrochloride, DIPEA, ACN, reflux, 3 h; (b) zinc powder, HOAc, EtOH, 0° C. to rt, overnight; (c) NaOH, 30% H₂O₂ aq, H₂O, MeOH, reflux, 3 h; (d) SOCl₂, DMF, 100° C., 8 h; (e) 4-(ethoxycarbonylmethyl)phenylboronic acid pinacol ester, Pd(PPh₃)₄, Cs₂CO₃, 1,4-dioxane, H₂O, 85° C., overnight; (f) 4-(2-(4-(4,4,5,5-tetramethyl-1,3,2-dioxaborolan-2-yl)-1H-pyrazol-1-yl)ethyl) morpholine, Pd₂ (dba)₃, PCy₃, Na₂CO₃, H₂O, DMF, 120° C., overnight; (g) NaOH, THF, H₂O, 0° C. to rt, 16 h; (h) EDC·HCl, HOAt, TEA, DMF, DMA, 0° C. to rt, overnight.

### Step a: Synthesis of Ethyl (5-bromo-2-nitrophenyl)glycinate (2) (ZW-10-189)

A solution of 4-bromo-2-fluoro-1-nitrobenzene (1, 20.0 g, 90.0 mmol), glycine ether ester hydrochloride (14.0 g, 100.0 mmol), and N, N-diisopropylethylamine (23.6 mL, 136.0 mmol) in acetonitrile (400.0 mL) was refluxed for 3 hours. After cooling, the reaction mixture was concentrated under vacuum to remove the majority of acetonitrile. Then the residue was diluted with water (100.0 mL) and extracted with ethyl acetate (100.0 mL*5). The combined organic layers were washed with brine, dried under anhydrous sodium sulfate, filtered, concentrated under vacuum to afford a yellow solid as ethyl (5-bromo-2-nitrophenyl)glycinate (2, 25.2 g, 92.4% yields). ¹H NMR (400 MHZ, CDCl₃) δ 8.49-8.36 (m, 1H), 8.05 (d, J=8.9 Hz, 1H), 6.87-6.79 (m, 2H), 4.29 (q, J=7.1 Hz, 2H), 4.05 (d, J=5.1 Hz, 2H), 1.32 (t, J=7.1 Hz, 3H). ESI-MS m/z [M+H]⁺ 303.

### Step b: Synthesis of Ethyl (5-bromo-2-nitrophenyl)glycinate (3) (ZW-10-191)

Acetic acid (23.8 mL, 415.7 mmol) diluted with ethanol (50.0 mL) was added dropwise to a suspension of zinc powder (27.2 g, 415.7 mmol) and ethyl (5-bromo-2-nitrophenyl)glycinate (2, 25.2 g, 83.1 mmol) in ethanol (150.0 mL) at 0° C. over a course of 1 hour. After addition, the reaction mixture was allowed to stir at room temperature overnight. LC-MS analysis indicated the completed conversion. After filtration, the filtrate was concentrated under vacuum to afford a beige foam as ethyl 2-((2-amino-5-bromophenyl)amino) acetate (3), which was used directly for next step without further purifications. ESI-MS m/z [M+H]⁺ 273.

### Step c: Synthesis of 6-bromoquinoxalin-2 (1/I)-one (4) (ZW-10-197)

Ethyl 2-((2-amino-5-bromophenyl)amino) acetate (3, 8.3 g, 30.4 mmol) was dissolved in a mixture of water and methanol (60.0 mL, v/v=4:1). Sodium hydroxide (7.3 g, 182.4 mmol) was added to the reaction mixture followed by 30% hydrogen peroxide (5.2 mL, 45.6 mmol). The reaction mixture was heated to reflux for 5 hours. Then the resulting mixture was cooled in an ice bath and slowly acidified to pH=5 with 2 N hydrochloric acid aqueous solution. The precipitate was collected, washed with water and ether, and dried under vacuum to afford a light brown solid as 6-bromoquinoxalin-2 (1H)-one. (4, 6.5 g, 95.2% yields). ¹H NMR (400 MHZ, DMSO-d₆) δ 12.51 (s, 1H), 8.19 (s, 1H), 7.96 (s, 1H), 7.70 (d, J=8.7 Hz, 1H), 7.25 (d, J=8.7 Hz, 1H). ESI-MS m/z [M+H]⁺ 225.

### Step d: Synthesis of 6-bromo-2-chloroquinoxaline (5) (ZW-11-002)

6-bromoquinoxalin-2 (1H)-one (4, 5.0 g, 22.3 mmol) was slowly added to iced phosphorus oxychloride (33.3 mL, 357.3 mmol) to give a red slurry. To the resulting slurry was added dropwise dimethylformamide (3.4 mL, 24.6 mmol) below 15° C. After addition, the red mixture was refluxed for 15 minutes to generate a clear solution. After cooling back to room temperature, the solution was slowly added to iced water (200.0 mL), and the mixture was basified slowly with 40% sodium hydroxide aqueous solution to pH 8~9. The resulting solid was collected by filtration, washed with water (50.0 mL) and dried under vacuum to afford a light-yellow solid as 6-bromo-2-chloroquinoxaline (5, 4.2 g, 77.8% yields). ¹H NMR (400 MHZ, CDCl₃) δ 8.76 (s, 1H), 8.29 (s, 1H), 7.93-7.85 (m, 2H). ESI-MS m/z [M+H]⁺ 243.

### Step e: Synthesis of Ethyl 2-(4-(6-bromoquinoxalin-2-yl)phenyl) acetate (6) (ZW-2-007) (ZW-11-011)

To a 20.0 mL microwave reaction vial were added 6-bromo-2-chloroquinoxaline (5, 6.0 g, 24.6 mmol), 4-(ethoxycarbonylmethyl)phenylboronic acid pinacol ester (6.5 g, 22.2 mmol), cesium carbonate (18.2 g, 56.0 mmol), tetrakis(triphenylphosphine) palladium (0) (2.6 g, 2.2 mmol), 1,4-dioxane (100.0 mL), and water (10.0 mL). After degassing with N₂ for 15 minutes, the reaction mixture was sealed and stirred at 85° C. overnight. After cooling, the reaction mixture was concentrated under vacuum, absorbed onto silica gel, and purified via flash chromatography (ethyl acetate:hexanes=1:99 to 1:10) to afford a off-white foam as ethyl 2-(4-(6-bromoquinoxalin-2-yl)phenyl) acetate (6, 4.9 g, 59.1% yields). 1H NMR (400 MHZ, CDCl₃) δ 9.30 (s, 1H), 8.28 (s, 1H), 8.15 (d, J=8.5 Hz, 2H), 7.99 (d, J=8.9 Hz, 1H), 7.91-7.77 (m, 1H), 7.48 (d, J=8.6 Hz, 2H), 4.17 (q, J=7.1 Hz, 2H), 3.70 (s, 2H), 1.26 (t, J=7.1 Hz, 3H). ESI-MS m/z [M+H]⁺ 371.

### Step f: Synthesis of Ethyl 2-(4-(6-(1-(2-morpholinoethyl)-1H-pyrazol-4-yl) quinoxalin-2-yl)phenyl) acetate (7) (ZW-11-014)

To a 20.0 mL microwave reaction vial were added ethyl 2-(4-(6-bromoquinoxalin-2-yl)phenyl) acetate (6, 4.9 g, 13.2 mmol), 4-(2-(4-(4,4,5,5-tetramethyl-1,3,2-dioxaborolan-2-yl)-1H-pyrazol-1-yl)ethyl) morpholine (6.1 g, 19.9 mmol), tris(dibenzylideneacetone) dipalladium (0) (87.3 mg, 0.1 mmol), tricyclohexylphosphine (0.1 g, 0.4 mmol), sodium carbonate (7.0 g, 66.2 mmol), water (1.7 mL) and dimethylformamide (16.7 mL). After degassing with N₂ for 30 minutes, the reaction mixture was sealed and stirred at 120° C. overnight. After cooling, the reaction mixture was concentrated under vacuum, absorbed onto silica gel, and purified via flash chromatography (ethyl acetate:hexanes=1:99 to 1:1, 0.5% triethylamine in ethyl acetate) to afford a yellow foam as ethyl 2-(4-(6-(1-(2-morpholinoethyl)-1H-pyrazol-4-yl) quinoxalin-2-yl)phenyl) acetate (7, 4.2 g, 67.3% yields). ¹H NMR (400 MHZ, CDCl₃) δ 9.28 (s, 1H), 8.17-8.09 (m, 4H), 7.95-7.89 (m, 3H), 7.48 (d, J=6.5 Hz, 2H), 4.32 (t, J=6.4 Hz, 2H), 4.17 (q, J=7.1 Hz, 2H), 3.73-3.69 (m, 6H), 2.88 (t, J=6.3 Hz, 2H), 2.56-2.48 (m, 4H), 1.26 (t, J=7.1 Hz, 3H). ESI-MS m/z [M+H]⁺ 472.

### Step g: Synthesis of 2-(4-(6-(1-(2-morpholinoethyl)-1H-pyrazol-4-yl) quinoxalin-2-yl)phenyl) acetic Acid (8) (ZW-10-185)

To a solution of ethyl 2-(4-(6-(1-(2-morpholinoethyl)-1H-pyrazol-4-yl) quinoxalin-2-yl)phenyl) acetate (7, 2.4 g, 5.0 mmol) in tetrahydrofuran (60.0 mL) was added a fresh prepared solution of sodium hydroxide (0.6 g, 15.0 mmol) in water (10.0 mL) in a ice-bath. Then the reaction mixture was allowed to stir at room temperature for 16 hours. LC-MS analysis indicated the completed conversion. The reaction mixture was concentrated under vacuum to remove the majority of tetrahydrofuran. The residue was diluted with water (20.0 mL) and acidified to pH 4 with 4 N hydrochloric acid aqueous solution. The resulting mixture was concentrated under vacuum to afford a brown solid as the crude product of 2-(4-(6-(1-(2-morpholinoethyl)-1H-pyrazol-4-yl) quinoxalin-2-yl)phenyl) acetic acid (8) which was used directly for next step without further purifications. ESI-MS m/z [M+H]⁺ 444.

### Step h: Synthesis of N-(5-(tert-butyl) isoxazol-3-yl)-2-(4-(6-(1-(2-morpholinoethyl)-1H-pyrazol-4-yl) quinoxalin-2-yl)phenyl) acetamide (9) (ZW-10-193)

A mixture of 2-(4-(6-(1-(2-morpholinoethyl)-1H-pyrazol-4-yl) quinoxalin-2-yl)phenyl) acetic acid (8, 4.9 mmol), 1-(3-dimethylaminopropyl)-3-ethylcarbodiimide hydrochloride (2.4 g, 12.2 mmol), 1 N 1-hydroxy-7-azabenzotriazole in dimethylacetamide (4.9 mL, 4.9 mmol), and triethylamine (4.1 mL, 29.4 mmol) in dimethylformamide (40.0 mL) were stirred at 0° C. for 15 minutes. Then 5-(tert-butyl) isoxazol-3-amine (1.0 g, 7.4 mmol) was added in one portion at 0° C. and the resulting mixture was stirred at room temperature overnight. The resulting mixture was diluted with water (200.0 mL) and extracted with ethyl acetate (200.0 mL*3). The combined organic layers were washed with brine, dried over anhydrous sodium sulfate, filtered, condensed under vacuum, absorbed onto silica gel, and purified via flash chromatography (dichloromethane:methanol=99:1 to 20:1, 0.5% triethylamine in methanol) to afford a yellow foam as the crude product. The crude product was further purified via C18 reversed-phase flash column chromatography (deionized water: methanol=95:5 to 100% MeOH) to afford an off-white solid as N-(5-(tert-butyl) isoxazol-3-yl)-2-(4-(6-(1-(2-morpholinoethyl)-1H-pyrazol-4-yl) quinoxalin-2-yl)phenyl) acetamide (9, 1.6 g, 57.7% yields). ¹H NMR (400 MHZ, CDCl₃) δ 9.27 (s, 1H), 8.74 (s, 1H), 8.22-8.10 (m, 4H), 7.96-7.90 (m, 3H), 7.53 (d, J=6.8 Hz, 2H), 6.71 (s, 1H), 4.32 (t, J=6.5 Hz, 2H), 3.85 (s, 2H), 3.75-3.67 (m, 4H), 2.88 (t, J=6.4 Hz, 2H), 2.52 (t, J=4.2 Hz, 4H), 1.33 (s, 9H). ¹³C NMR (101 MHZ, CDCl₃) δ 181.90, 168.50, 157.59, 150.41, 143.52, 142.18, 141.23, 137.22, 136.32, 135.62, 134.20, 130.19 (2C), 130.05, 128.79, 128.09 (2C), 127.37, 123.68, 121.88, 93.25, 66.90 (2C), 58.15, 53.69 (2C), 49.94, 43.99, 33.04, 28.61 (3C). ESI-MS m/z [M+H]⁺ 566. HPLC purity: 97.37% (gradient mobile phase: deionized water with 0.1% v/v triethylamine (solvent A): MeOH with 0.1% v/v triethylamine (solvent B): =from 3:7 to 100% solvent B, flow rate: 0.8 mL/min, run-time: 12.00 min, retention time: 9.97 min).

### REFERENCES

1 Siegel R L, Miller K D, Jemal A. Cancer statistics, 2020. CA: A Cancer Journal for Clinicians 2020; 70 (1): 7-30 doi 10.3322/caac.21590.
2. Genomic and Epigenomic Landscapes of Adult De Novo Acute Myeloid Leukemia. New England Journal of Medicine 2013; 368 (22): 2059-74 doi 10.1056/NEJMoa1301689.
3. Thiede C, Steudel C, Mohr B, Schaich M, Schakel U, Platzbecker U, et al. Analysis of FLT3-activating mutations in 979 patients with acute myelogenous leukemia: association with FAB subtypes and identification of subgroups with poor prognosis. Blood 2002; 99 (12): 4326-35.
4. Spiekermann K, Bagrintseva K, Schoch C, Haferlach T, Hiddemann W, Schnittger S. A new and recurrent activating length mutation in exon 20 of the FLT3 gene in acute myeloid leukemia. Blood 2002; 100 (9): 3423-5 doi 10.1182/blood-2002-03-0953.
5. Smith C C, Wang Q, Chin C-S, Salerno S, Damon L E, Levis M J, et al. Validation of ITD mutations in FLT3 as a therapeutic target in human acute myeloid leukaemia. Nature 2012; 485 (7397): 260-3 doi 10.1038/nature11016.
6 Antar A I, Otrock Z K, Jabbour E, Mohty M, Bazarbachi A. FLT3 inhibitors in acute myeloid leukemia: ten frequently asked questions. Leukemia 2020; 34 (3): 682-96 doi 10.1038/s41375-019-0694-3.
7. Stone R M, Mandrekar S J, Sanford B L, Laumann K, Geyer S, Bloomfield C D, et al. Midostaurin plus Chemotherapy for Acute Myeloid Leukemia with a FLT3 Mutation. New England Journal of Medicine 2017; 377 (5): 454-64 doi 10.1056/NEJMoa1614359.
8. Levis M. Midostaurin approved for FLT3-mutated AML. Blood 2017; 129 (26): 3403-6 doi 10.1182/blood-2017-05-782292.
9 Fischer T, Stone R M, DeAngelo D J, Galinsky I, Estey E, Lanza C, et al. Phase IIB Trial of Oral Midostaurin (PKC412), the FMS-Like Tyrosine Kinase 3 Receptor (FLT3) and Multi-Targeted Kinase Inhibitor, in Patients With Acute Myeloid Leukemia and High-Risk Myelodysplastic Syndrome With Either Wild-Type or Mutated FLT3. Journal of Clinical Oncology 2010; 28 (28): 4339-45 doi 10.1200/JCO.2010.28.9678.
10. Cortes J E, Kantarjian H, Foran J M, Ghirdaladze D, Zodelava M, Borthakur G, et al. Phase I Study of Quizartinib Administered Daily to Patients With Relapsed or Refractory Acute Myeloid Leukemia Irrespective of FMS-Like Tyrosine Kinase 3-Internal Tandem Duplication Status. Journal of Clinical Oncology 2013; 31 (29): 3681-7 doi 10.1200/JCO.2013.48.8783.
11. Zorn J A, Wang Q, Fujimura E, Barros T, Kuriyan J. Crystal Structure of the FLT3 Kinase Domain Bound to the Inhibitor Quizartinib (AC220). PLOS ONE 2015; 10 (4): e0121177 doi 10.1371/journal.pone.0121177.
12. Klug L R, Kent J D, Heinrich M C. Structural and clinical consequences of activation loop mutations in class III receptor tyrosine kinases. Pharmacology & Therapeutics 2018; 191:123-34.
13. Mackarehtschian K, Hardin J D, Moore K A, Boast S, Goff S P, Lemischka I R. Targeted disruption of the flk2/flt3 gene leads to deficiencies in primitive hematopoietic progenitors. Immunity 1995; 3 (1): 147-61.
14. Warkentin A A, Lopez M S, Lasater E A, Lin K, He B-L, Leung A Y H, et al. Overcoming myelosuppression due to synthetic lethal toxicity for FLT3-targeted acute myeloid leukemia therapy. eLife 2014; 3: e03445 doi 10.7554/eLife.03445.
15. Lee L Y, Hernandez D, Rajkhowa T, Smith S C, Raman J R, Nguyen B, et al. Preclinical studies of gilteritinib, a next-generation FLT3 inhibitor. Blood 2017; 129 (2): 257-60 doi 10.1182/blood-2016-10-745133.
16. Yamaura T, Nakatani T, Uda K, Ogura H, Shin W, Kurokawa N, et al. A novel irreversible FLT3 inhibitor, FF-10101, shows excellent efficacy against AML cells with FLT3 mutations. Blood 2018; 131 (4): 426-38 doi 10.1182/blood-2017-05-786657.
17. Mori M, Kaneko N, Ueno Y, Yamada M, Tanaka R, Saito R, et al. Gilteritinib, a FLT3/AXL inhibitor, shows antileukemic activity in mouse models of FLT3 mutated acute myeloid leukemia. Investigational New Drugs 2017; 35 (5): 556-65 doi 10.1007/s10637-017-0470-z.
18. McMahon C M, Ferng T, Canaani J, Wang E S, Morrissette J J D, Eastburn D J, et al. Clonal selection with Ras pathway activation mediates secondary clinical resistance to selective FLT3 inhibition in acute myeloid leukemia. Cancer Discovery 2019: CD-18-1453 doi 10.1158/2159-8290. CD-18-1453.
19. Smith C C, Perl A E, Lasater E, Zhang C, Jeschke G R, Damon L E, et al. PLX3397 Is An 19 Investigational Selective FLT3 Inhibitor That Retains Activity Against the Clinically-Relevant FLT3-ITD/F691L “Gatekeeper” Mutation in Vitro. Blood 2011; 118 (21): 764-doi 10.1182/blood.V118.21.764.764.
20. Smith C C, Zhang C, Lin K C, Lasater E A, Zhang Y, Massi E, et al. Characterizing and Overriding the Structural Mechanism of the Quizartinib-Resistant FLT3 “Gatekeeper” F691L Mutation with PLX3397. Cancer Discovery 2015; 5 (6): 668-79 doi 10.1158/2159-8290. cd-15-0060.
21. Smith C C, Levis M J, Frankfurt O, Pagel J M, Roboz G J, Stone R M, et al. A phase 1/2 study of the oral FLT3 inhibitor pexidartinib in relapsed/refractory FLT3-ITD-mutant acute myeloid leukemia. Blood Advances 2020; 4 (8): 1711-21 doi 10.1182/bloodadvances.2020001449.
22. Bharate J B, McConnell N, Naresh G, Zhang L, Lakkaniga N R, Ding L, et al. Rational Design, Synthesis and Biological Evaluation of Pyrimidine-4,6-diamine derivatives as Type-II inhibitors of FLT3 Selective Against c-KIT. Scientific Reports 2018; 8 (1): 3722 doi 10.1038/s41598-018-21839-3.
23. Frett B, McConnell N, Smith C C, Wang Y, Shah N P, Li H-y. Computer aided drug discovery of highly ligand efficient, low molecular weight imidazopyridine analogs as FLT3 inhibitors. European journal of medicinal chemistry 2015; 94:123-31 doi 10.1016/j.ejmech.2015.02.052.
24. Carey A, Edwards D K V, Eide C A, Newell L, Traer E, Medeiros B C, et al. Identification of Interleukin-1 by Functional Screening as a Key Mediator of Cellular Expansion and Disease Progression in Acute Myeloid Leukemia. Cell Reports 2017; 18 (13): 3204-18 doi 10.1016/j.celrep.2017.03.018.
25. Apsel Winger B, Cortopassi W A, Garrido Ruiz D, Ding L, Jang K, Leyte-Vidal A, et al. ATP-competitive inhibitors midostaurin and avapritinib have distinct resistance profiles in exon 17-mutant KIT. Cancer Research 2019: canres.3139.2018 doi 10.1158/0008-5472. CAN-18-3139.
26. Musselman C A, Lalonde M-E, Côté J, Kutateladze T G. Perceiving the epigenetic landscape through histone readers. Nature structural & molecular biology 2012; 19 (12): 1218-27 doi 10.1038/nsmb.2436.
27. Taverna S D, Li H, Ruthenburg A J, Allis C D, Patel D J. How chromatin-binding modules interpret histone modifications: lessons from professional pocket pickers. Nature Structural & Molecular Biology 2007; 14 (11): 1025-40 doi 10.1038/nsmb1338.
28. Huang D, Zhou T, Lafleur K, Nevado C, Caflisch A. Kinase selectivity potential for inhibitors targeting the ATP binding site: a network analysis. Bioinformatics 2010; 26 (2): 198-204 doi 10.1093/bioinformatics/btp650.
29. Bower H, Björkholm M, Dickman P W, Höglund M, Lambert P C, Andersson T M L. Life Expectancy of Patients With Chronic Myeloid Leukemia Approaches the Life Expectancy of the General Population. Journal of Clinical Oncology 2016; 34 (24): 2851-7 doi 10.1200/JCO.2015.66.2866.
30. Branford S, Rudzki Z, Walsh S, Grigg A, Arthur C, Taylor K, et al. High frequency of point mutations clustered within the adenosine triphosphate-binding region of BCR/ABL in patients with chronic myeloid leukemia or Ph-positive acute lymphoblastic leukemia who develop imatinib (STI571) resistance. Blood 2002; 99 (9): 3472-5 doi 10.1182/blood. V99.9.3472.
31. Gore L, Kearns P R, de Martino M L, Lee, De Souza C A, Bertrand Y, et al. Dasatinib in Pediatric Patients With Chronic Myeloid Leukemia in Chronic Phase: Results From a Phase II Trial. Journal of Clinical Oncology 2018; 36 (13): 1330-8 doi 10.1200/JCO.2017.75.9597.
32. Shah N P, Tran C, Lee F Y, Chen P, Norris D, Sawyers C L. Overriding Imatinib Resistance with a Novel A BL Kinase Inhibitor. Science 2004; 305 (5682): 399 doi 10.1126/science.1099480.
33. Shah N P, Skaggs B J, Branford S, Hughes T P, Nicoll J M, Paquette R L, et al. Sequential ABL kinase inhibitor therapy selects for compound drug-resistant BCR-ABL mutations with altered oncogenic potency. The Journal of clinical investigation 2007; 117 (9): 2562-9 doi 10.1172/JCI30890.
34. Gorre M E, Mohammed M, Ellwood K, Hsu N, Paquette R, Rao P N, et al. Clinical Resistance to STI-571 Cancer Therapy Caused by BCR-ABL Gene Mutation or Amplification. Science 2001; 293 (5531): 876 doi 10.1126/science. 1062538.
35. Shah N P, Nicoll J M, Nagar B, Gorre M E, Paquette R L, Kuriyan J, et al. Multiple BCR-ABL kinase domain mutations confer polyclonal resistance to the tyrosine kinase inhibitor imatinib (STI571) in chronic phase and blast crisis chronic myeloid leukemia. Cancer Cell 2002; 2 (2): 117-25.
36. Schultz K R, Carroll A, Heerema N A, Bowman W P, Aledo A, Slayton W B, et al. Long-term follow-up of imatinib in pediatric Philadelphia chromosome-positive acute lymphoblastic leukemia: Children's Oncology Group study AALL0031. Leukemia 2014; 28 (7): 1467-71 doi 10.1038/leu.2014.30.
37. Napolitano A, Vincenzi B. Secondary KIT mutations: the GIST of drug resistance and sensitivity. British Journal of Cancer 2019; 120 (6): 577-8 doi 10.1038/s41416-019-0388-7.
38. Dibb N J, Dilworth S M, Mol C D. Switching on kinases: oncogenic activation of BRAF 38. and the PDGFR family. Nature Reviews Cancer 2004; 4 (9): 718-27 doi 10.1038/nrc1434.
39. Smith C C, Lasater E A, Lin K C, Wang Q, McCreery M Q, Stewart W K, et al. Crenolanib is a selective type I pan-FLT3 inhibitor. Proceedings of the National Academy of Sciences 2014; 111 (14): 5319 doi 10.1073/pnas.1320661111.
40. Levis M, Brown P, Smith B D, Stine A, Pham R, Stone R, et al. Plasma inhibitory activity (PIA): a pharmacodynamic assay reveals insights into the basis for cytotoxic response to FLT3 inhibitors. Blood 2006; 108 (10): 3477-83 doi 10.1182/blood-2006-04-015743.
41. Ma H S, Nguyen B, Duffield A S, Li L, Galanis A, Williams A B, et al. FLT3 Kinase Inhibitor TTT-3002 Overcomes Both Activating and Drug Resistance Mutations in FLT3 in Acute Myeloid Leukemia. Cancer Research 2014; 74 (18): 5206 doi 10.1158/0008-5472. CAN-14-1028.
42. Jacobson M P, Friesner R A, Xiang Z, Honig B. On the Role of the Crystal Environment in Determining Protein Side-chain Conformations. Journal of Molecular Biology 2002; 320 (3): 597-608.
43. Jacobson M P, Pincus D L, Rapp C S, Day T J F, Honig B, Shaw D E, et al. A hierarchical approach to all-atom protein loop prediction. Proteins: Structure, Function, and Bioinformatics 2004; 55 (2): 351-67.
44. Pettersen E F, Goddard T D, Huang C C, Meng E C, Couch G S, Croll T I, et al. UCSF ChimeraX: Structure visualization for researchers, educators, and developers. Protein Science 2021; 30 (1): 70-82.
45. Goddard T D, Huang C C, Meng E C, Pettersen E F, Couch G S, Morris J H, et al. UCSF ChimeraX: Meeting modern challenges in visualization and analysis. Protein Science 2018; 27 (1): 14-25.

1. A compound having a formula of 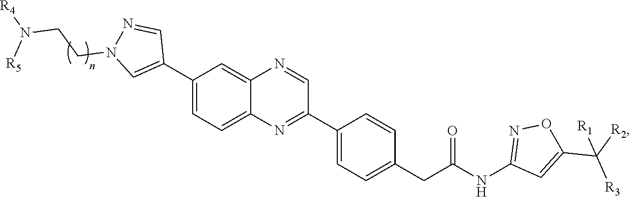
or a pharmaceutically acceptable salt thereof, wherein—
n is 1 or 2;
R¹, R², and R³ are independently selected from methyl optionally substituted with a halo; and
R⁴ and R⁵ together form a heterocyclic ring or C₁-C₃ alkyl.

2. The compound of claim 1, wherein R⁴ and R⁵ together form a morpholine ring.

3. The compound of any one of claims 1-2, wherein R¹, R², and R³ are methyl.

4. The compound of any one of claims 1-3, wherein n is 1.

5. The compound of claim 1 having a formula of

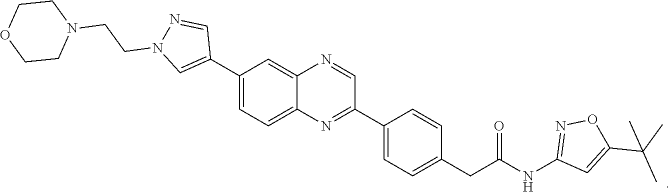 [SMILES: CC(C)(C)c1cc(NC(=O)Cc2ccc(-c3cnc4cc(-c5cnn(CCN6CCOCC6)c5)ccc4n3)cc2)no1]

6. A pharmaceutical composition comprising the compound of any one of claims 1-5 and a pharmaceutically acceptable excipient, carrier, or diluent.

7. A deuterated isotopologue of the compound according to any one of claims 1-5.

8. A pharmaceutical composition comprising the deuterated isotopologue of claim 7 and a pharmaceutically acceptable excipient, carrier, or diluent.

9. A method of treating a subject for a cell proliferative disorder comprising administering a FMS like tyrosine kinase 3 inhibitor to the subject.

10. The method of claim 9, wherein the cell proliferative disorder is a leukemia.

11. The method of claim 9, wherein the cell proliferative disorder is acute myeloid leukemia.

12. The method of claim 11, wherein the acute myeloid leukemia is characterized by a FLT3 mutation.

13. The method of claim 12, wherein the FLT3 mutation is a D835 mutation.

14. The method of claim 12, wherein the FLT3 mutation is a D835Y mutation.

15. The method of claim 12, wherein the FLT3 mutation is a F691 mutation.

16. The method of claim 12, wherein the FLT3 mutation is a F691L mutation.

17. The method of any one of claims 9-16, wherein the FMS like tyrosine kinase 3 inhibitor has a formula of 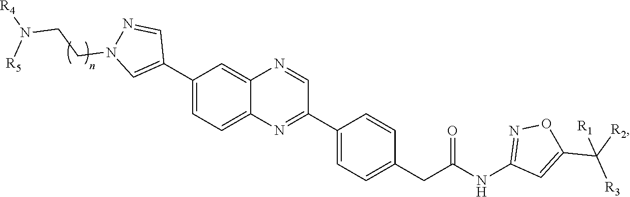
or a pharmaceutically acceptable salt thereof, wherein—
n is 1 or 2;
R¹, R², and R³ are independently selected from methyl optionally substituted with a halo; and
R⁴ and R⁵ together form a heterocyclic ring or C₁-C₃ alkyl.

18. The method of claim 17, wherein R⁴ and R⁵ together form a morpholine ring.

19. The method of any one of claims 17-18, wherein R¹, R², and R³ are methyl.

20. The method of any one of claims 17-19, wherein n is 1.

21. The method of claim 17, wherein the FMS like tyrosine kinase 3 inhibitor has a formula of

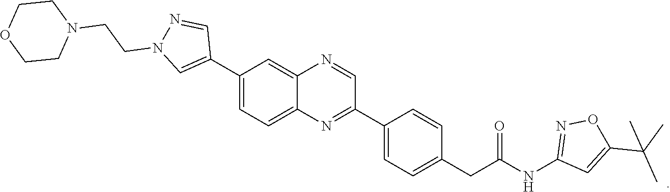 [SMILES: CC(C)(C)c1cc(NC(=O)Cc2ccc(-c3cnc4cc(-c5cnn(CCN6CCOCC6)c5)ccc4n3)cc2)no1]

22. The method of any one of claims 9-16, wherein the FMS like tyrosine kinase 3 inhibitor has a formula of

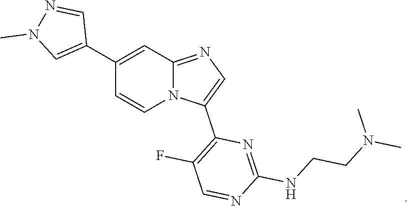

23. The method of any one of claims 9-22, wherein the FMS like tyrosine kinase 3 inhibitor is a deuterated isotopologue.

24. The method of any one of claims 9-23 further comprising administering a cell proliferative disorder chemotherapy.

25. The method of claim 24, wherein the cell proliferative disorder chemotherapy is an acute myeloid leukemia chemotherapy.

26. A method of making a compound, the method comprising:
providing a compound of Formula I 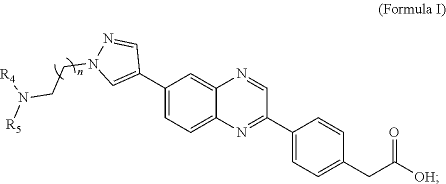
and converting the compound of Formula I into a compound having a formula of 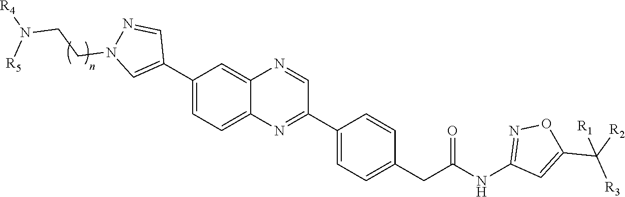
or a pharmaceutically acceptable salt thereof, wherein—
n is 1 or 2;
R¹, R², and R³ are independently selected from methyl optionally substituted with a halo; and
R⁴ and R⁵ together form a heterocyclic ring or C₁-C₃ alkyl.

27. The method of claim 26, wherein providing the compound of formula I comprises providing a compound of Formula II 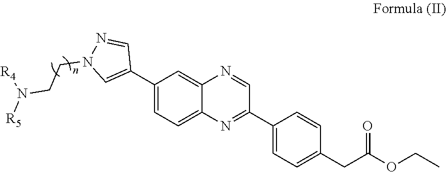
and converting the compound of Formula II into the compound of Formula I.

28. The method of claim 27, wherein providing the compound of Formula II comprises providing a compound of Formula III 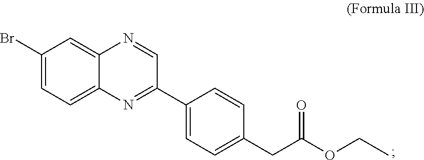 [SMILES: CCOC(=O)Cc1ccc(-c2cnc3cc(Br)ccc3n2)cc1]
and converting the compound of Formula III into the compound of Formula II.
